(19) United States
(12) Patent Application Publication
(10) Pub. No.: US 2015/0380656 A1
(43) Pub. Date: Dec. 31, 2015

(54) MATERIAL FOR ORGANIC ELECTROLUMINESCENCE DEVICE AND ORGANIC ELECTROLUMINESCENCE DEVICE USING THE SAME

(71) Applicant: SAMSUNG DISPLAY CO., LTD., Yongin-City (KR)

(72) Inventor: Naoya Sakamoto, Yokohama (JP)

(21) Appl. No.: 14/706,171

(22) Filed: May 7, 2015

(30) Foreign Application Priority Data
Jun. 30, 2014 (JP) 2014-133854

(51) Int. Cl. H01L 51/00; C07C 211/58; C07D 307/91; C07D 333/76; C09K 11/06; C07C 211/61; C07C 211/56; C07C 255/58; C07C 217/80; C07D 213/36; C07C 211/54; C07F 7/08

(52) U.S. Cl. CPC H01L 51/0059; C07C 211/54; C07C 211/58; C07D 307/91; C07D 333/76; C07F 7/081; C07C 211/61; C07C 211/56; C07C 255/58; C07C 217/80; C07D 213/36; C09K 11/06; H01L 51/006; H01L 51/0061; H01L 51/0094; H01L 51/0067; C09K 2211/1014; C09K 2211/1007; C09K 2211/1011; C09K 2211/1088; C09K 2211/1092; C09K 2211/1029; H01L 51/0073; H01L 51/0074; H01L 51/0052; H01L 51/0054; H01L 2251/308; H01L 51/5206

(57) ABSTRACT

A compound for an organic electroluminescence device and an organic electroluminescence device, the compound being represented by the following Chemical Formula 1:

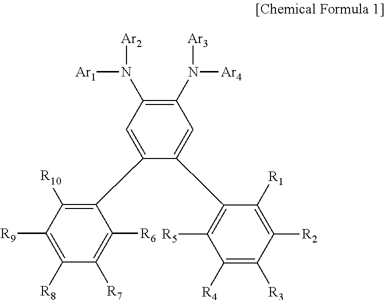

### CROSS-REFERENCE TO RELATED APPLICATION

Japanese Patent Application No. 2014-133854, filed on Jun. 30, 2014, in the Japanese Patent Office, and entitled: “Material for Organic Electroluminescence Device and Organic Electroluminescence Device Using the Same,” is incorporated by reference herein in its entirety.

### BACKGROUND

1. Field

Embodiments relate to a material for an organic electroluminescence device and an organic electroluminescence device using the same.

2. Description of the Related Art

In recent years, organic electroluminescence displays have been actively developed. An organic electroluminescence device that is a self-luminescent device used in the organic electroluminescence displays has been actively developed.

An organic electroluminescence device may have a structure that includes a positive electrode, a hole transport layer disposed on the positive electrode, an emission layer disposed on the hole transport layer, an electron transport layer disposed on the emission layer, and a negative electrode disposed on the electron transport layer.

In the organic electroluminescence device, holes and electrons injected from the positive electrode and the negative electrode are recombined in the emission layer to generate excitons and transit the generated excitons to a ground state, thereby emitting light.

### SUMMARY

Embodiments are directed to a material for an organic electroluminescence device and an organic electroluminescence device using the same.

The embodiments may be realized by providing a compound for an organic electroluminescence device, the compound being represented by the following Chemical Formula 1:

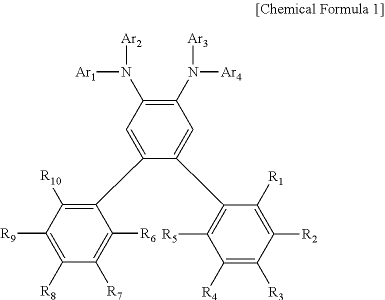

wherein, in Chemical Formula 1, Ar₁ to Ar₄ are each independently a substituted or unsubstituted aryl group or a substituted or unsubstituted heteroaryl group, at least one of Ar₁ to Ar₄ is a substituted or unsubstituted aryl group having 10 or more ring carbon atoms or a substituted or unsubstituted heteroaryl group having 10 or more ring carbon atoms, R₁ to R₁₀ are each independently a hydrogen atom, a substituted or unsubstituted aryl group, a substituted or unsubstituted heteroaryl group, a substituted or unsubstituted alkyl group, a substituted or unsubstituted alkoxy group, a halogen atom, an electron suction group, or a deuterium atom, and adjacent ones of R₁ to R₁₀ are separate or are combined to each other to form a saturated or unsaturated ring.

The aryl group having 10 or more ring carbon atoms may include a biphenyl group, a naphthyl group, a phenanthrenyl group, or a triphenylenyl group.

The aryl group having 10 or more ring carbon atoms may include the biphenyl group.

The heteroaryl group having 10 or more ring carbon atoms may include a dibenzohetero group.

The dibenzohetero group may be a group represented by the following Chemical Formula 2:

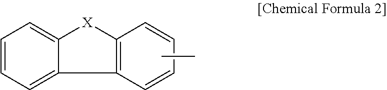

wherein, in Chemical Formula 2, X may be O, S, or Si(R₁₁)₂, and R₁₁ may be a substituted or unsubstituted alkyl group or a substituted or unsubstituted aryl group.

Ar₁ to Ar₄ may each independently be a substituted or unsubstituted aryl group having 10 or more ring carbon atoms or a substituted or unsubstituted heteroaryl group having 10 or more ring carbon atoms.

The embodiments may be realized by providing an organic electroluminescence device, including a compound for an organic electroluminescence device, the compound being represented by the following Chemical Formula 1:

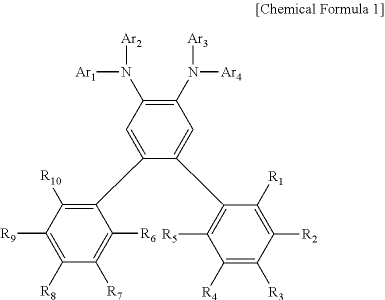

wherein, in Chemical Formula 1, Ar₁ to Ar₄ are each independently a substituted or unsubstituted aryl group or a substituted or unsubstituted heteroaryl group, at least one of Ar₁ to Ar₄ is a substituted or unsubstituted aryl group having 10 or more ring carbon atoms or a substituted or unsubstituted heteroaryl group having 10 or more ring carbon atoms, R₁ to R₁₀ are each independently a hydrogen atom, a substituted or unsubstituted aryl group, a substituted or unsubstituted heteroaryl group, a substituted or unsubstituted alkyl group, a substituted or unsubstituted alkoxy group, a halogen atom, an electron withdrawing group, or a deuterium atom, and adjacent ones of R₁ to R₁₀ are separate or are combined to each other to form a saturated or unsaturated ring.

The aryl group having 10 or more ring carbon atoms may include a biphenyl group, a naphthyl group, a phenanthrenyl group, or a triphenylenyl group.

The aryl group having 10 or more ring carbon atoms may include a biphenyl group.

The heteroaryl group having 10 or more ring carbon atoms may include a dibenzohetero group.

The dibenzohetero group may be a group represented by the following Chemical Formula 2:

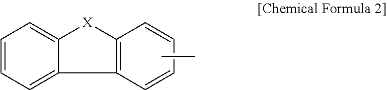

wherein, in Chemical Formula 2, X may be O, S, or Si(R₁₁)₂, and R₁₁ may be a substituted or unsubstituted alkyl group or a substituted or unsubstituted aryl group.

Ar₁ to Ar₄ may each independently be a substituted or unsubstituted aryl group having 10 or more ring carbon atoms or a substituted or unsubstituted heteroaryl group having 10 or more ring carbon atoms.

The compound for an organic electroluminescence device may include one of the following Compounds 1-1 to 1-24:

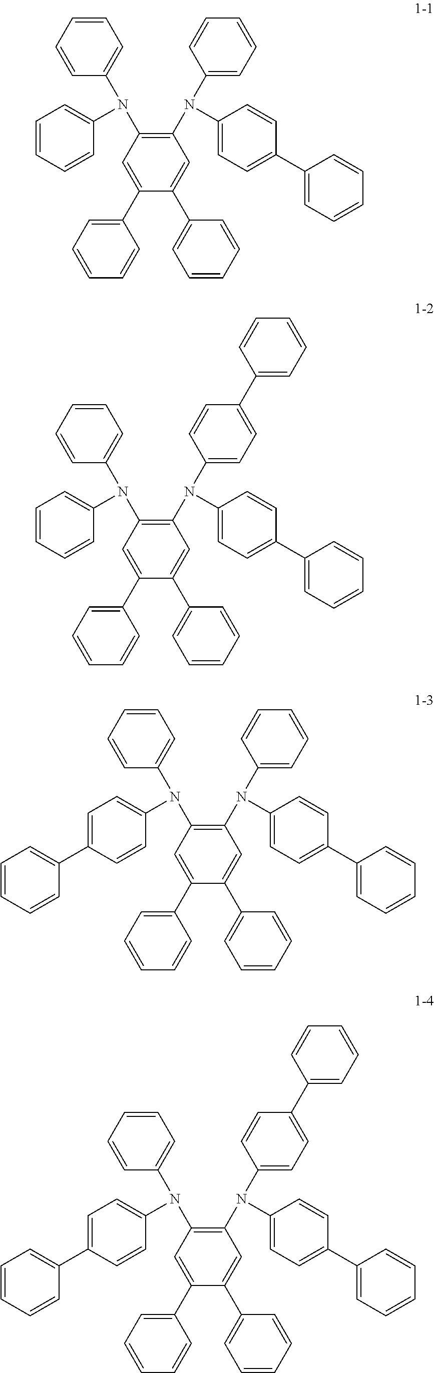

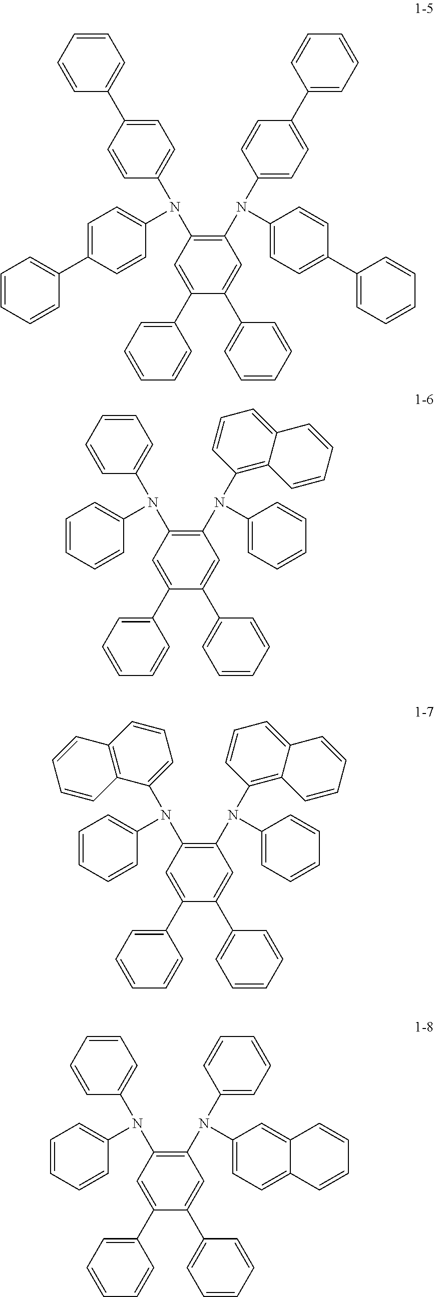

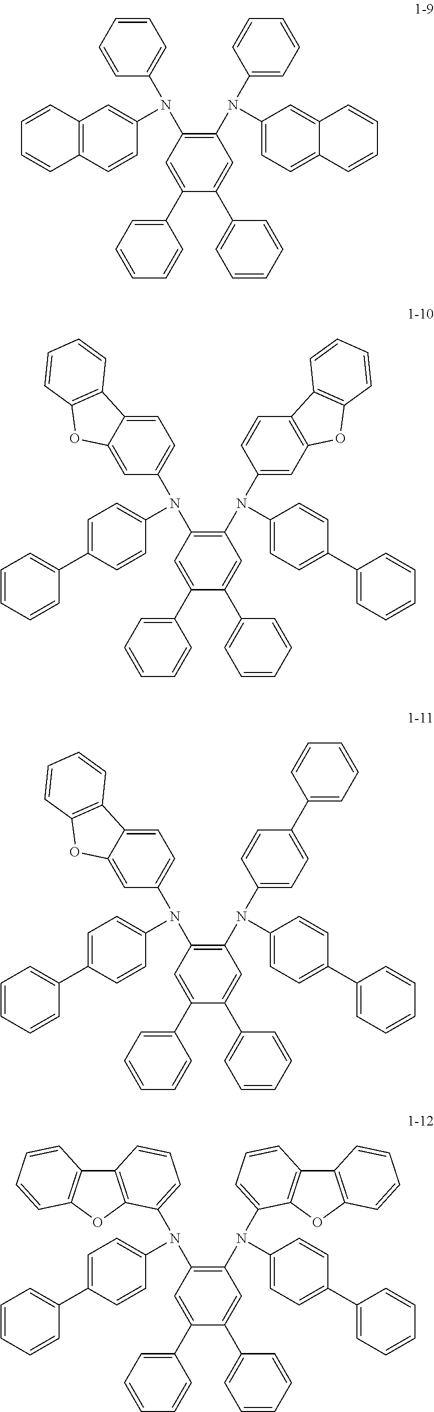 [SMILES: c1ccc(-c2cc(N(c3ccccc3)c3ccc4ccccc4c3)c(N(c3ccccc3)c3ccc4ccccc4c3)cc2-c2ccccc2)cc1 ; c1ccc(-c2ccc(N(c3ccc4c(c3)oc3ccccc34)c3cc(-c4ccccc4)c(-c4ccccc4)cc3N(c3ccc(-c4ccccc4)cc3)c3ccc4c(c3)oc3ccccc34)cc2)cc1 ; c1ccc(-c2ccc(N(c3ccc(-c4ccccc4)cc3)c3cc(-c4ccccc4)c(-c4ccccc4)cc3N(c3ccc(-c4ccccc4)cc3)c3ccc4c(c3)oc3ccccc34)cc2)cc1 ; c1ccc(-c2ccc(N(c3cc(-c4ccccc4)c(-c4ccccc4)cc3N(c3ccc(-c4ccccc4)cc3)c3cccc4c3oc3ccccc34)c3cccc4c3oc3ccccc34)cc2)cc1]

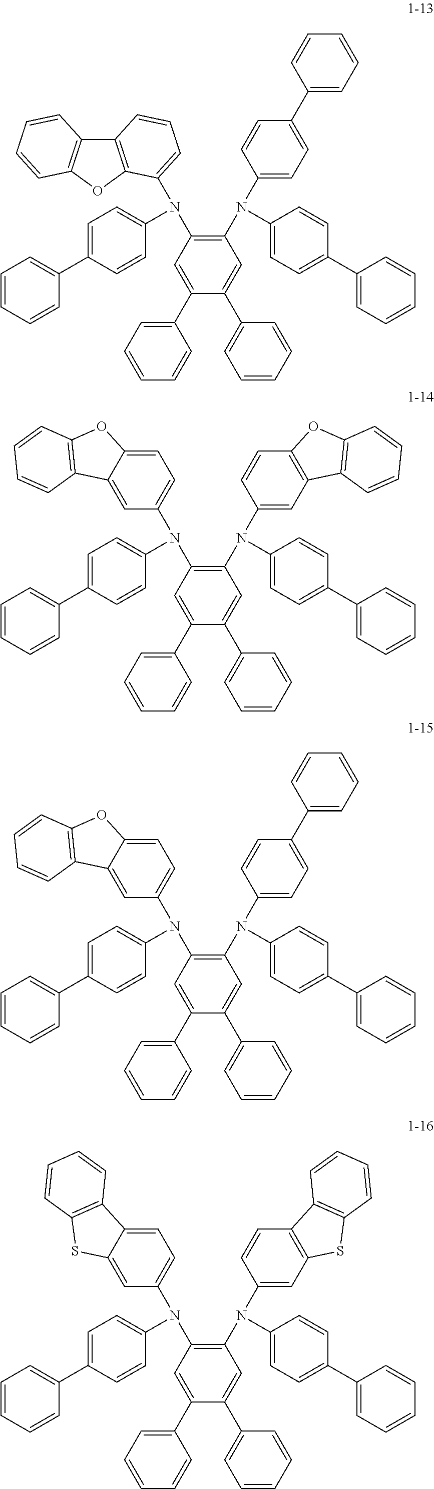 [SMILES: c1ccc(-c2ccc(N(c3ccc(-c4ccccc4)cc3)c3cc(-c4ccccc4)c(-c4ccccc4)cc3N(c3ccc(-c4ccccc4)cc3)c3cccc4c3oc3ccccc34)cc2)cc1 ; c1ccc(-c2ccc(N(c3ccc4oc5ccccc5c4c3)c3cc(-c4ccccc4)c(-c4ccccc4)cc3N(c3ccc(-c4ccccc4)cc3)c3ccc4oc5ccccc5c4c3)cc2)cc1 ; c1ccc(-c2ccc(N(c3ccc(-c4ccccc4)cc3)c3cc(-c4ccccc4)c(-c4ccccc4)cc3N(c3ccc(-c4ccccc4)cc3)c3ccc4oc5ccccc5c4c3)cc2)cc1 ; c1ccc(-c2ccc(N(c3ccc4c(c3)sc3ccccc34)c3cc(-c4ccccc4)c(-c4ccccc4)cc3N(c3ccc(-c4ccccc4)cc3)c3ccc4c(c3)sc3ccccc34)cc2)cc1]

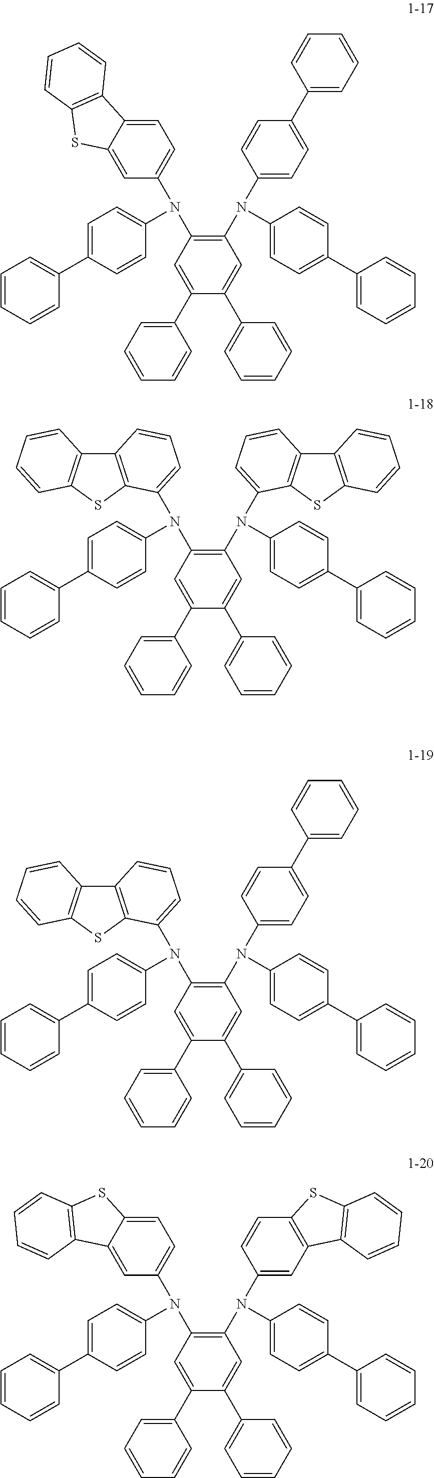 [SMILES: c1ccc(-c2ccc(N(c3ccc(-c4ccccc4)cc3)c3cc(-c4ccccc4)c(-c4ccccc4)cc3N(c3ccc(-c4ccccc4)cc3)c3ccc4c(c3)sc3ccccc34)cc2)cc1 ; c1ccc(-c2ccc(N(c3cc(-c4ccccc4)c(-c4ccccc4)cc3N(c3ccc(-c4ccccc4)cc3)c3cccc4c3sc3ccccc34)c3cccc4c3sc3ccccc34)cc2)cc1 ; c1ccc(-c2ccc(N(c3ccc(-c4ccccc4)cc3)c3cc(-c4ccccc4)c(-c4ccccc4)cc3N(c3ccc(-c4ccccc4)cc3)c3cccc4c3sc3ccccc34)cc2)cc1 ; c1ccc(-c2ccc(N(c3ccc4sc5ccccc5c4c3)c3cc(-c4ccccc4)c(-c4ccccc4)cc3N(c3ccc(-c4ccccc4)cc3)c3ccc4sc5ccccc5c4c3)cc2)cc1]

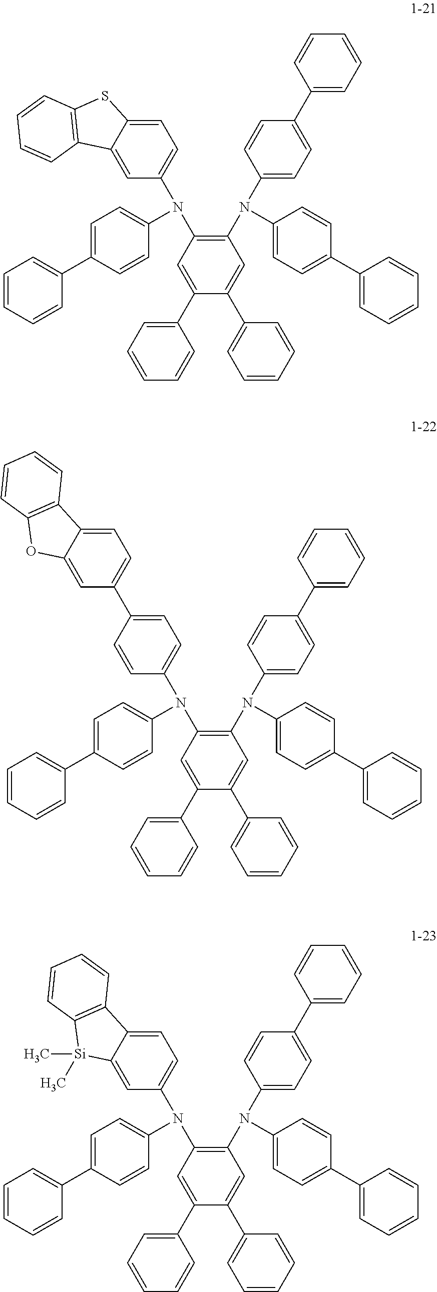 [SMILES: c1ccc(-c2ccc(N(c3ccc(-c4ccccc4)cc3)c3cc(-c4ccccc4)c(-c4ccccc4)cc3N(c3ccc(-c4ccccc4)cc3)c3ccc4sc5ccccc5c4c3)cc2)cc1 ; c1ccc(-c2ccc(N(c3ccc(-c4ccccc4)cc3)c3cc(-c4ccccc4)c(-c4ccccc4)cc3N(c3ccc(-c4ccccc4)cc3)c3ccc(-c4ccc5c(c4)oc4ccccc45)cc3)cc2)cc1 ; C[Si]1(C)c2ccccc2-c2ccc(N(c3ccc(-c4ccccc4)cc3)c3cc(-c4ccccc4)c(-c4ccccc4)cc3N(c3ccc(-c4ccccc4)cc3)c3ccc(-c4ccccc4)cc3)cc21]

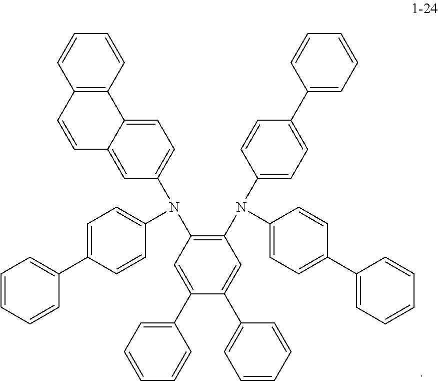 [SMILES: c1ccc(-c2ccc(N(c3ccc(-c4ccccc4)cc3)c3cc(-c4ccccc4)c(-c4ccccc4)cc3N(c3ccc(-c4ccccc4)cc3)c3ccc4c(ccc5ccccc54)c3)cc2)cc1]

The compound for an organic electroluminescence device may include one of the following Compounds 1-25 to 1-44:

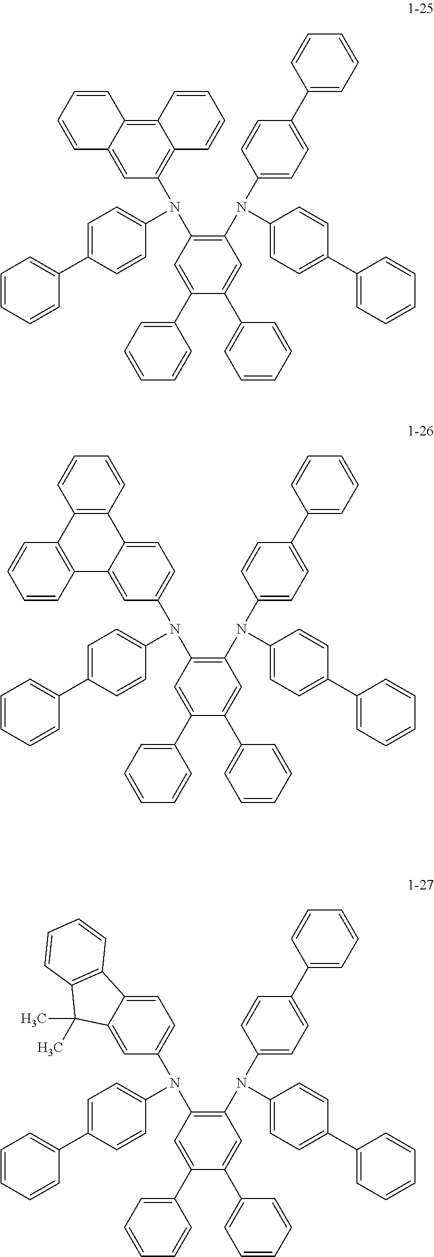 [SMILES: c1ccc(-c2ccc(N(c3ccc(-c4ccccc4)cc3)c3cc(-c4ccccc4)c(-c4ccccc4)cc3N(c3ccc(-c4ccccc4)cc3)c3cc4ccccc4c4ccccc34)cc2)cc1 ; c1ccc(-c2ccc(N(c3ccc(-c4ccccc4)cc3)c3cc(-c4ccccc4)c(-c4ccccc4)cc3N(c3ccc(-c4ccccc4)cc3)c3ccc4c5ccccc5c5ccccc5c4c3)cc2)cc1 ; CC1(C)c2ccccc2-c2ccc(N(c3ccc(-c4ccccc4)cc3)c3cc(-c4ccccc4)c(-c4ccccc4)cc3N(c3ccc(-c4ccccc4)cc3)c3ccc(-c4ccccc4)cc3)cc21]

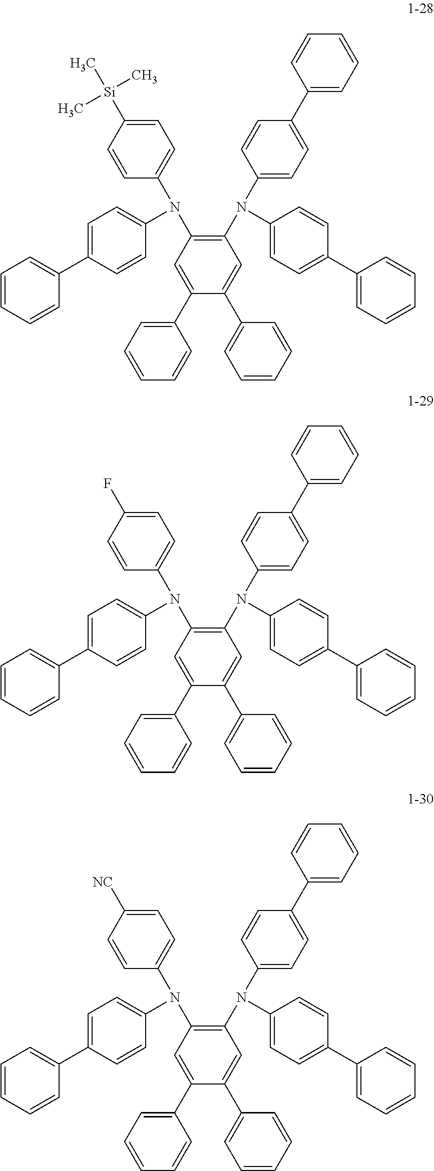 [SMILES: C[Si](C)(C)c1ccc(N(c2ccc(-c3ccccc3)cc2)c2cc(-c3ccccc3)c(-c3ccccc3)cc2N(c2ccc(-c3ccccc3)cc2)c2ccc(-c3ccccc3)cc2)cc1 ; Fc1ccc(N(c2ccc(-c3ccccc3)cc2)c2cc(-c3ccccc3)c(-c3ccccc3)cc2N(c2ccc(-c3ccccc3)cc2)c2ccc(-c3ccccc3)cc2)cc1 ; N#Cc1ccc(N(c2ccc(-c3ccccc3)cc2)c2cc(-c3ccccc3)c(-c3ccccc3)cc2N(c2ccc(-c3ccccc3)cc2)c2ccc(-c3ccccc3)cc2)cc1]

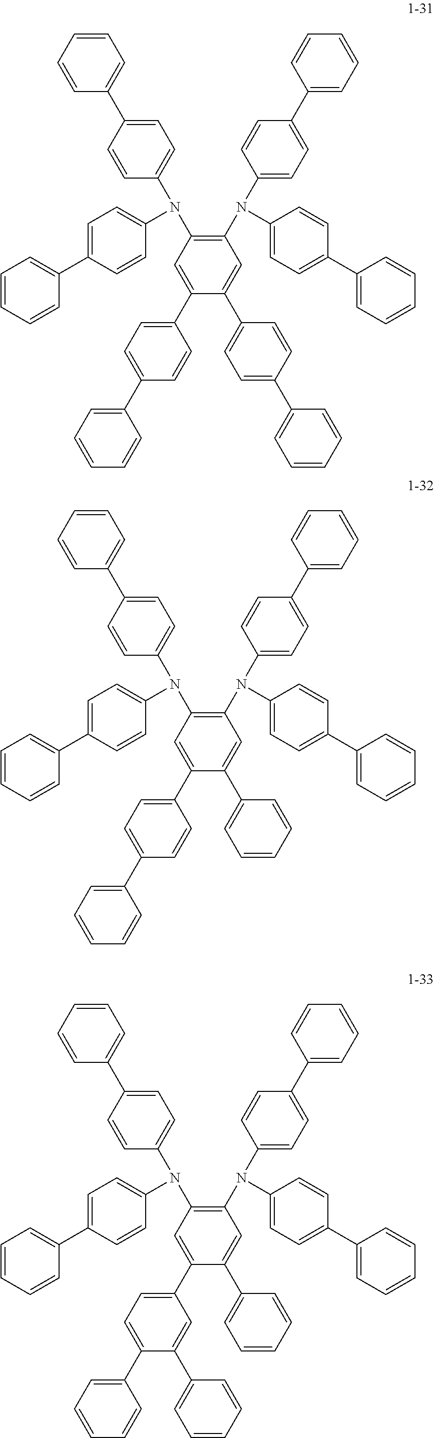 [SMILES: c1ccc(-c2ccc(-c3cc(N(c4ccc(-c5ccccc5)cc4)c4ccc(-c5ccccc5)cc4)c(N(c4ccc(-c5ccccc5)cc4)c4ccc(-c5ccccc5)cc4)cc3-c3ccc(-c4ccccc4)cc3)cc2)cc1 ; c1ccc(-c2ccc(-c3cc(N(c4ccc(-c5ccccc5)cc4)c4ccc(-c5ccccc5)cc4)c(N(c4ccc(-c5ccccc5)cc4)c4ccc(-c5ccccc5)cc4)cc3-c3ccccc3)cc2)cc1 ; c1ccc(-c2ccc(N(c3ccc(-c4ccccc4)cc3)c3cc(-c4ccccc4)c(-c4ccc(-c5ccccc5)c(-c5ccccc5)c4)cc3N(c3ccc(-c4ccccc4)cc3)c3ccc(-c4ccccc4)cc3)cc2)cc1]

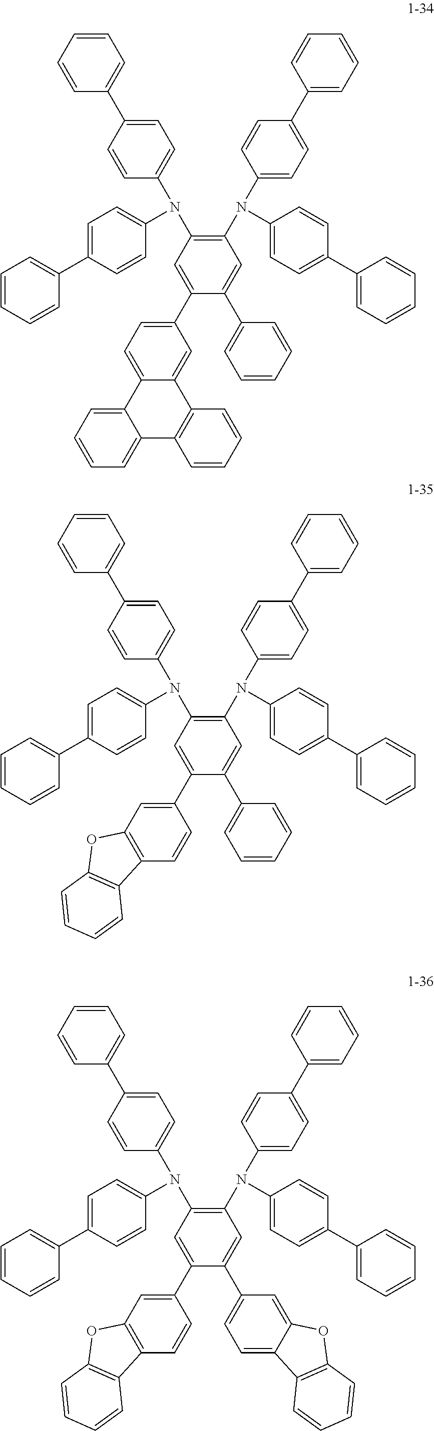 [SMILES: c1ccc(-c2ccc(N(c3ccc(-c4ccccc4)cc3)c3cc(-c4ccccc4)c(-c4ccc5c6ccccc6c6ccccc6c5c4)cc3N(c3ccc(-c4ccccc4)cc3)c3ccc(-c4ccccc4)cc3)cc2)cc1 ; c1ccc(-c2ccc(N(c3ccc(-c4ccccc4)cc3)c3cc(-c4ccccc4)c(-c4ccc5c(c4)oc4ccccc45)cc3N(c3ccc(-c4ccccc4)cc3)c3ccc(-c4ccccc4)cc3)cc2)cc1 ; c1ccc(-c2ccc(N(c3ccc(-c4ccccc4)cc3)c3cc(-c4ccc5c(c4)oc4ccccc45)c(-c4ccc5c(c4)oc4ccccc45)cc3N(c3ccc(-c4ccccc4)cc3)c3ccc(-c4ccccc4)cc3)cc2)cc1]

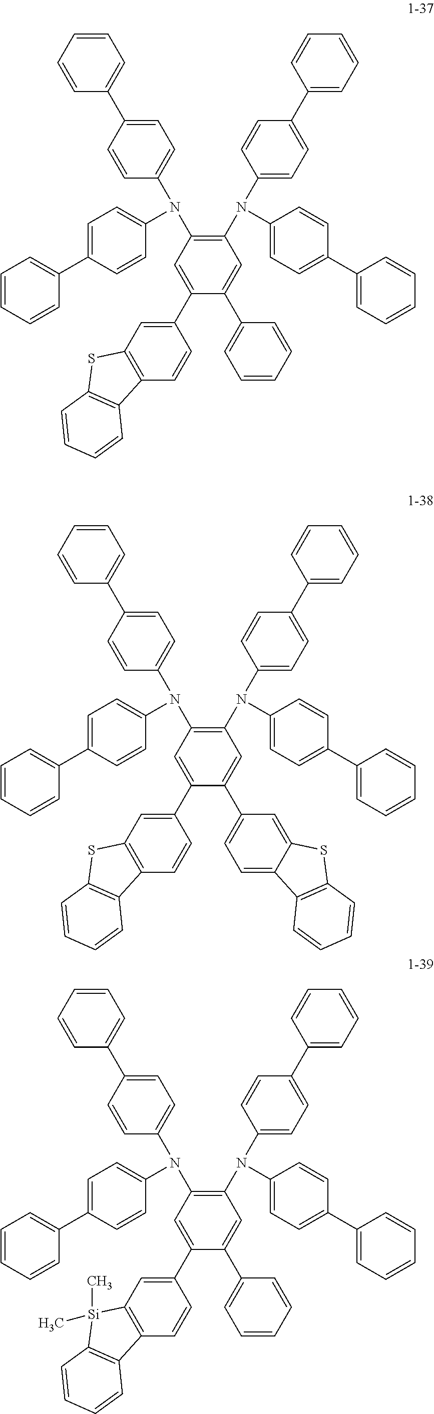

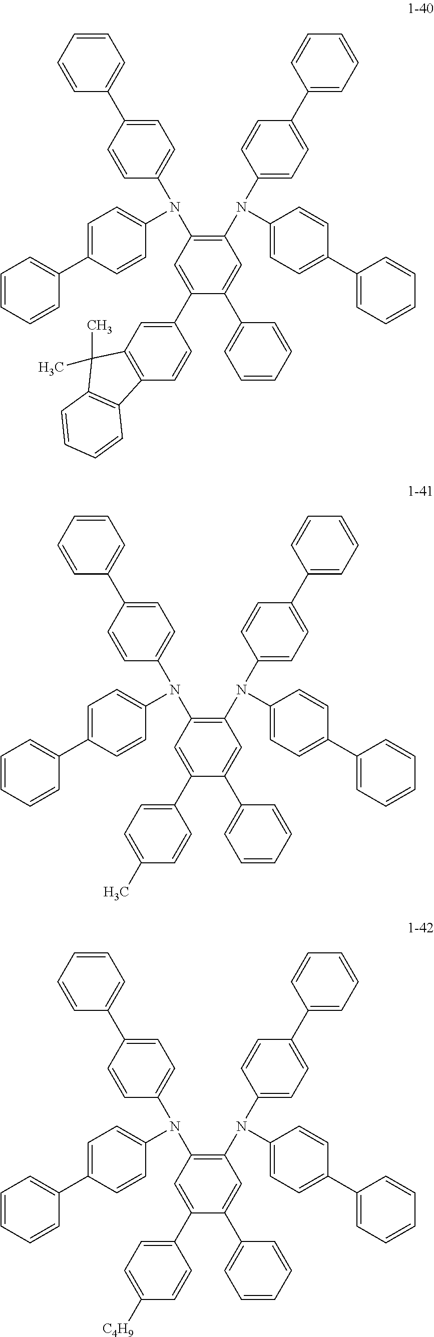

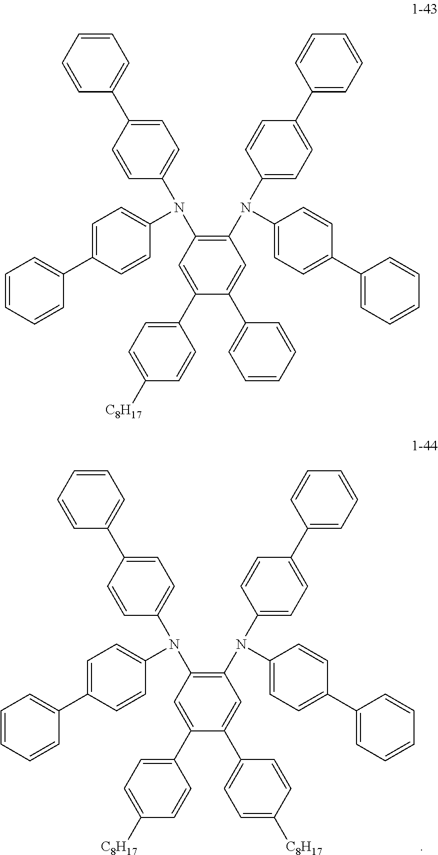

The compound for an organic electroluminescence device may include one of the following Compounds 1-45 to 1-58:

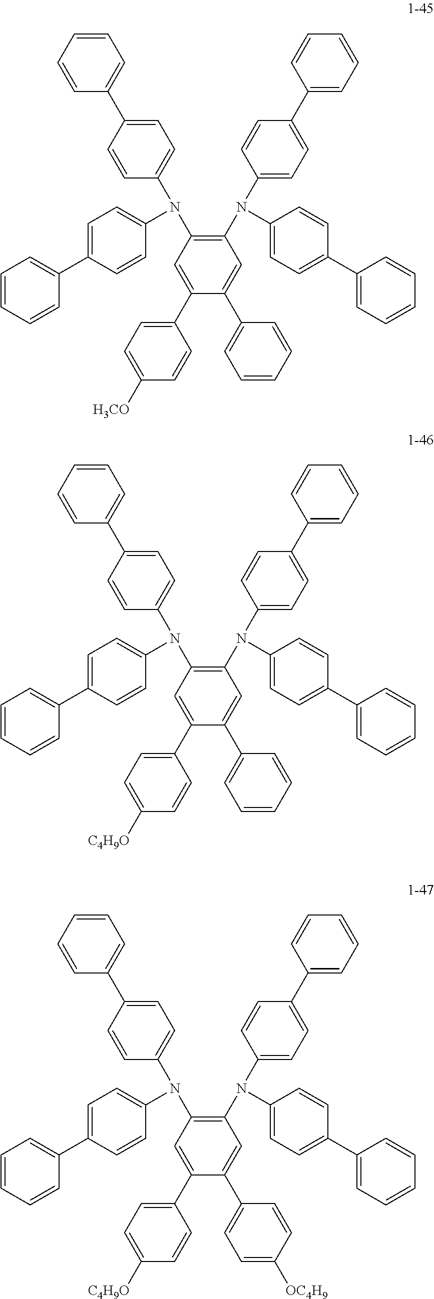

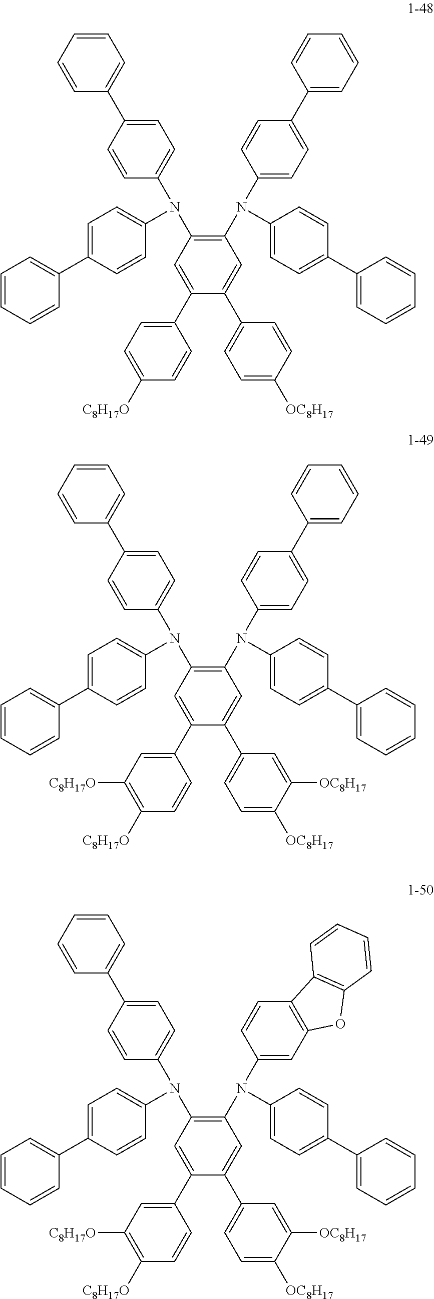

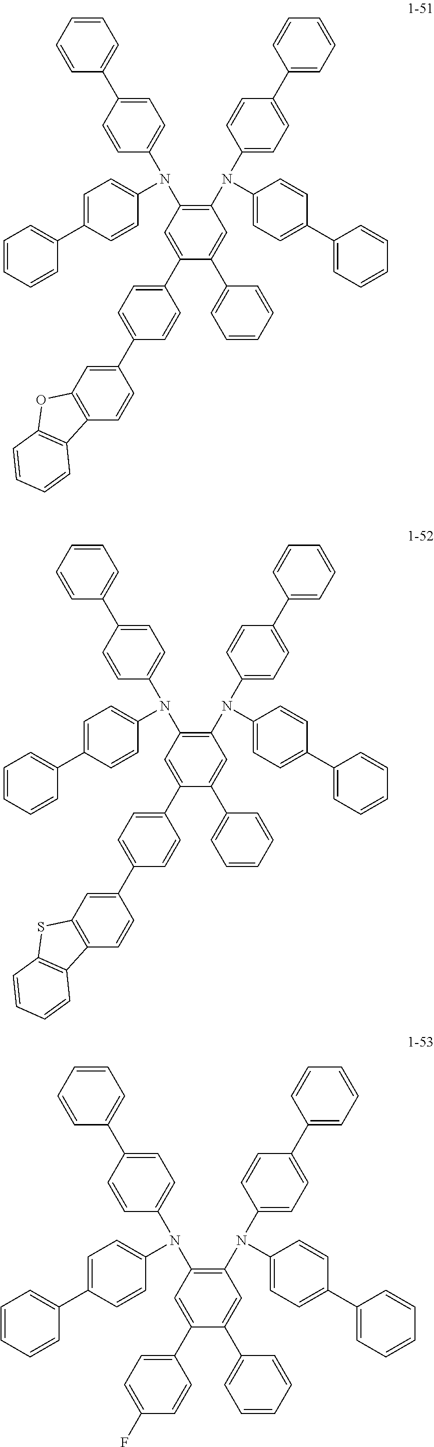 [SMILES: c1ccc(-c2ccc(N(c3ccc(-c4ccccc4)cc3)c3cc(-c4ccccc4)c(-c4ccc(-c5ccc6c(c5)oc5ccccc56)cc4)cc3N(c3ccc(-c4ccccc4)cc3)c3ccc(-c4ccccc4)cc3)cc2)cc1 ; c1ccc(-c2ccc(N(c3ccc(-c4ccccc4)cc3)c3cc(-c4ccccc4)c(-c4ccc(-c5ccc6c(c5)sc5ccccc56)cc4)cc3N(c3ccc(-c4ccccc4)cc3)c3ccc(-c4ccccc4)cc3)cc2)cc1 ; Fc1ccc(-c2cc(N(c3ccc(-c4ccccc4)cc3)c3ccc(-c4ccccc4)cc3)c(N(c3ccc(-c4ccccc4)cc3)c3ccc(-c4ccccc4)cc3)cc2-c2ccccc2)cc1]

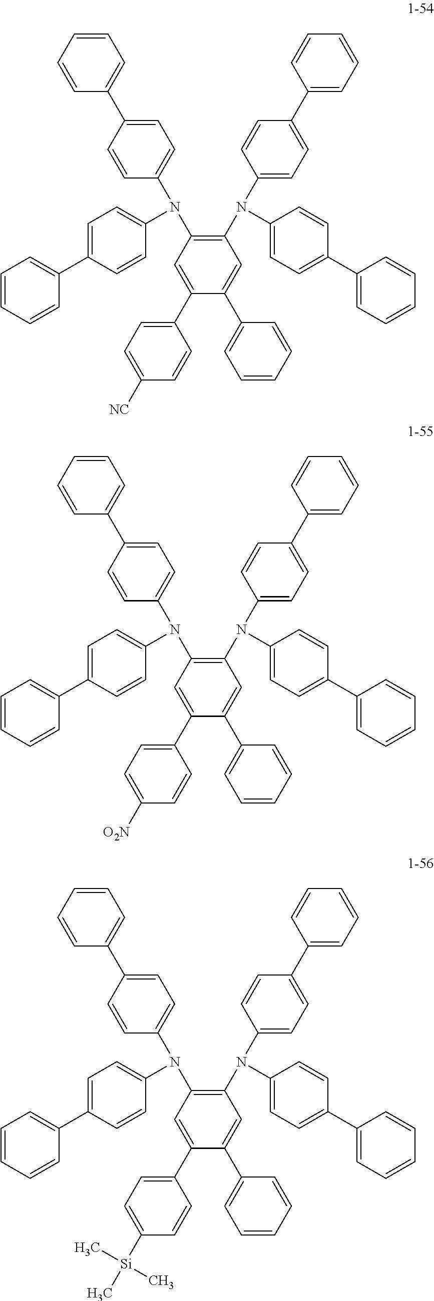 [SMILES: N#Cc1ccc(-c2cc(N(c3ccc(-c4ccccc4)cc3)c3ccc(-c4ccccc4)cc3)c(N(c3ccc(-c4ccccc4)cc3)c3ccc(-c4ccccc4)cc3)cc2-c2ccccc2)cc1 ; O=[N+]([O-])c1ccc(-c2cc(N(c3ccc(-c4ccccc4)cc3)c3ccc(-c4ccccc4)cc3)c(N(c3ccc(-c4ccccc4)cc3)c3ccc(-c4ccccc4)cc3)cc2-c2ccccc2)cc1 ; C[Si](C)(C)c1ccc(-c2cc(N(c3ccc(-c4ccccc4)cc3)c3ccc(-c4ccccc4)cc3)c(N(c3ccc(-c4ccccc4)cc3)c3ccc(-c4ccccc4)cc3)cc2-c2ccccc2)cc1]

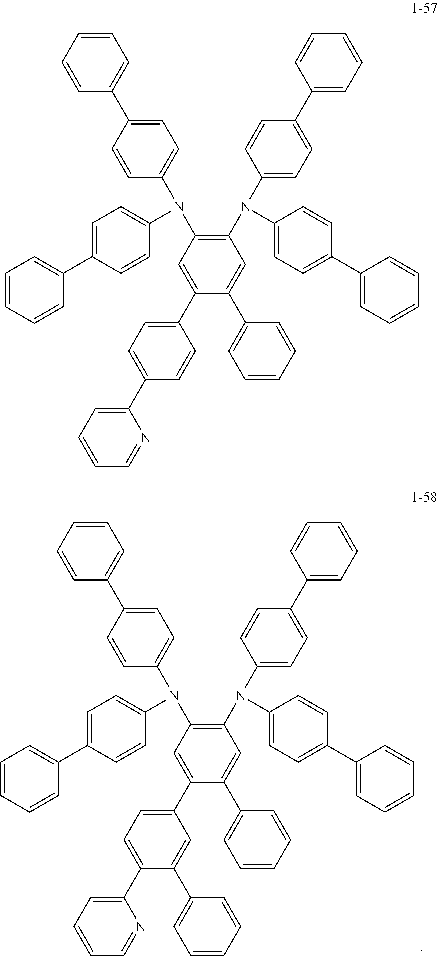 [SMILES: c1ccc(-c2ccc(N(c3ccc(-c4ccccc4)cc3)c3cc(-c4ccccc4)c(-c4ccc(-c5ccccn5)cc4)cc3N(c3ccc(-c4ccccc4)cc3)c3ccc(-c4ccccc4)cc3)cc2)cc1 ; c1ccc(-c2ccc(N(c3ccc(-c4ccccc4)cc3)c3cc(-c4ccccc4)c(-c4ccc(-c5ccccn5)c(-c5ccccc5)c4)cc3N(c3ccc(-c4ccccc4)cc3)c3ccc(-c4ccccc4)cc3)cc2)cc1]

The organic electroluminescence device may include a hole transport layer, and the compound for an organic electroluminescence device may be included in the hole transport layer.

### BRIEF DESCRIPTION OF THE DRAWING

Features will be apparent to those of skill in the art by describing in detail exemplary embodiments with reference to the attached drawing in which:

FIG. 1 illustrates a cross-sectional view of a schematic configuration of an organic light-emitting device according to an embodiment.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. Like reference numerals refer to like elements throughout.

<1. Configuration of Compound for Organic Electroluminescence Device>

A compound for an organic electroluminescence device according to an embodiment may be capable of improving a driving voltage, emission efficiency, and an emission life of the organic electroluminescence device. For example, in the case where the compound for an organic electroluminescence device is used as a hole transport material, the driving voltage, emission efficiency, and the emission life of the organic electroluminescence device may be improved. First, the configuration of the compound for an organic electroluminescence device according to the present embodiment will be described. The compound for an organic electroluminescence device according to the present embodiment may be represented by the following Chemical Formula 1.

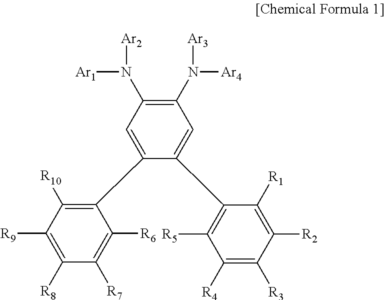

In Chemical Formula 1, Ar₁ to Ar₄ may each independently be or include, e.g., a substituted or unsubstituted aryl group or a substituted or unsubstituted heteroaryl group.

Examples of the aryl group and the heteroaryl group may include a phenyl group, a biphenyl group, a terphenyl group, a naphthyl group, an anthryl group, a phenanthryl group, a fluorenyl group, an indenyl group, a pyrenyl group, a fluoranthenyl group, a triphenylenyl group, a pyridyl group, a furanyl group, a pyranyl group, a thienyl group, a quinolyl group, an isoquinolyl group, a benzofuranyl group, a benzothienyl group, an indolyl group, a benzoxazolyl group, a benzothiazolyl group, a quinoxalyl group, a benzoxazolyl group, a pyrazolyl group, a dibenzohetero group, and the like. In an implementation, the aryl group and the heteroaryl group may include, e.g., a phenyl group, a biphenyl group, a naphthyl group, a dibenzohetero group, a phenanthrenyl group, or a triphenylenyl group. In an implementation, the dibenzohetero group may be a group represented by the following Chemical Formula 2. In Chemical Formula 2, X may be, e.g., one of O, S, or Si(R₁₁)₂. R₁₁ may be or may include, e.g., a substituted or unsubstituted alkyl group (such as a methyl group, an ethyl group, or the like). In an implementation, R₁₁ may be or may include, e.g., a substituted or unsubstituted aryl group (such as a phenyl group or the like).

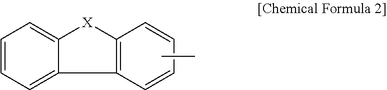

In an implementation, in Chemical Formula 1, at least one of Ar₁ to Ar₄ may be or may include, e.g., a substituted or unsubstituted aryl group having 10 or more ring carbon atoms or a substituted or unsubstituted heteroaryl group having 10 or more ring carbon atoms. Examples of the aryl group and the heteroaryl group having 10 or more ring carbon atoms may include the groups having 10 or more ring carbon atoms among the aforementioned examples of the aryl group and the heteroaryl group. In an implementation, the aryl group having 10 or more ring carbon atoms or the heteroaryl group having 10 or more ring carbon atoms may include, e.g., a biphenyl group, a naphthyl group, a dibenzohetero group, a phenanthrenyl group, or a triphenylenyl group. In an implementation, the aryl group having 10 or more ring carbon atoms or the heteroaryl group having 10 or more ring carbon atoms may include, e.g., a biphenyl group or a dibenzohetero group. In an implementation, all of Ar₁ to Ar₄ may be or include the substituted or unsubstituted aryl group having 10 or more ring carbon atoms or the substituted or unsubstituted heteroaryl group having 10 or more ring carbon atoms. In this case, stability of a cationic radical may be improved.

Examples of substituents of the aryl group and the heteroaryl group of Ar₁ to Ar₄ may include an alkyl group (for example, a methyl group, an ethyl group, and the like), a halogen atom (for example, a fluorine atom, a chlorine atom, and the like), a silyl group (for example, a trimethylsilyl group), a cyano group, an alkoxy group (for example, a methoxy group, a butoxy group, an octoxy group), a nitro group, a hydroxy group, a thiol group, and the like.

R₁ to R₁₀ may each independently be or include, e.g., a hydrogen atom, a substituted or unsubstituted aryl group, a substituted or unsubstituted heteroaryl group, a substituted or unsubstituted alkyl group (for example, a methyl group, an ethyl group, and the like), a substituted or unsubstituted alkoxy group (for example, a methoxy group, a butoxy group, and an octoxy group), a halogen atom (for example, a fluorine atom, a chlorine atom, and the like), an electron suction or withdrawing group (for example, a nitro group, and the like), or a deuterium atom. In an implementation, adjacent ones of R₁ to R₁₀ may be separate or may be combined to each other to form a saturated or unsaturated ring.

Examples of the aryl group and the heteroaryl group of R₁ to R₁₀ may include the same examples as the aryl group and the heteroaryl group of Ar₁ to Ar₄. Examples of substituents of the aryl group, the heteroaryl group, the alkyl group, and the alkoxy group of R₁ to R₁₀ may include an alkyl group (for example, a methyl group), a halogen atom (for example, a fluorine atom), a silyl group (for example, a trimethylsilyl group), a cyano group, an alkoxy group (for example, a methoxy group, a butoxy group, and an octoxy group), a nitro group, a hydroxy group, a thiol group, and the like.

In an implementation, the compound for an organic electroluminescence device according to the present embodiment may be included in at least one of a hole transport layer and an emission layer (among layers configuring the organic electroluminescence device). In an implementation, the compound for an organic electroluminescence device may be included in the hole transport layer, e.g., as a hole transport material.

In the organic electroluminescence device including the compound for an organic electroluminescence device having the aforementioned configuration, as will be described in the Examples below, a driving voltage, emission efficiency, and an emission life may be significantly improved. As the reason, without being bound by theory, the following two points may be considered.

First, a HOMO level of the compound for an organic electroluminescence device according to the present embodiment may be low. For example, as will be described in the Examples below, the HOMO level of the compound for an organic electroluminescence device according to the present embodiment may be about −5.69 eV or less. With respect to this, the HOMO level of another hole transport material may be about −5.3 eV. Accordingly, by including the compound for an organic electroluminescence device according to the present embodiment in the hole transport layer, holes may be smoothly injected from a hole injection layer to the hole transport layer.

Second, the compound for an organic electroluminescence device according to the present embodiment may have a large molecular weight, because at least one of Ar₁-Ar₄ may be the aryl group having 10 or more ring carbon atoms or the heteroaryl group having 10 or more ring carbon atoms. Therefore, a glass transition point may be high. In addition, the glass transition point of the material for an organic electroluminescence device may be increased, and thermal stability of the material may be increased. In addition, a film quality may be improved, and a long life or high efficiency of the device may be achieved. In an implementation, the compound for an organic electroluminescence device may include one of the following Compounds 1-1 to 1-58.

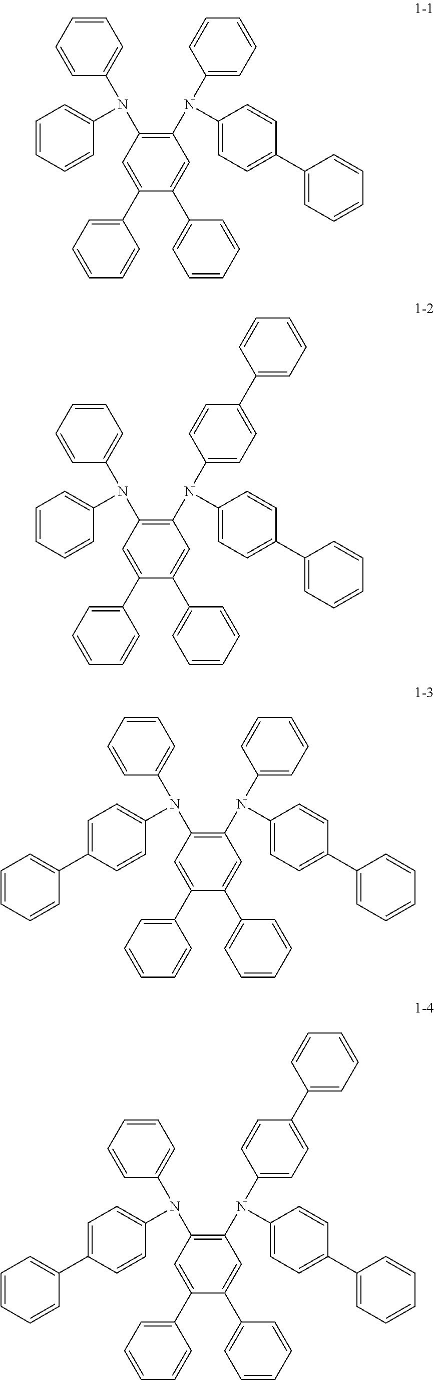 [SMILES: c1ccc(-c2ccc(N(c3ccccc3)c3cc(-c4ccccc4)c(-c4ccccc4)cc3N(c3ccccc3)c3ccccc3)cc2)cc1 ; c1ccc(-c2ccc(N(c3ccc(-c4ccccc4)cc3)c3cc(-c4ccccc4)c(-c4ccccc4)cc3N(c3ccccc3)c3ccccc3)cc2)cc1 ; c1ccc(-c2ccc(N(c3ccccc3)c3cc(-c4ccccc4)c(-c4ccccc4)cc3N(c3ccccc3)c3ccc(-c4ccccc4)cc3)cc2)cc1 ; c1ccc(-c2ccc(N(c3ccccc3)c3cc(-c4ccccc4)c(-c4ccccc4)cc3N(c3ccc(-c4ccccc4)cc3)c3ccc(-c4ccccc4)cc3)cc2)cc1]

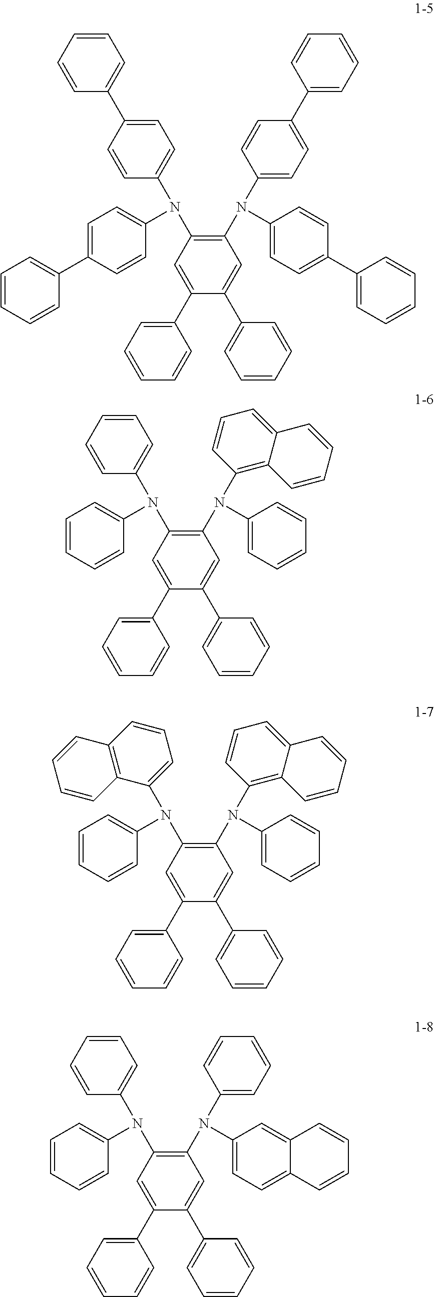

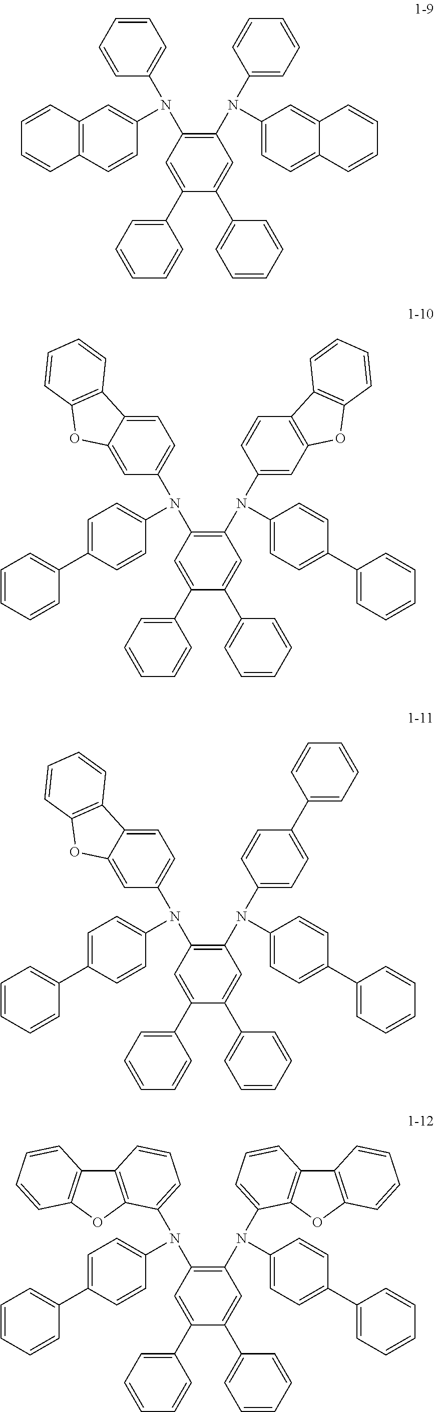 [SMILES: c1ccc(-c2cc(N(c3ccccc3)c3ccc4ccccc4c3)c(N(c3ccccc3)c3ccc4ccccc4c3)cc2-c2ccccc2)cc1 ; c1ccc(-c2ccc(N(c3ccc4c(c3)oc3ccccc34)c3cc(-c4ccccc4)c(-c4ccccc4)cc3N(c3ccc(-c4ccccc4)cc3)c3ccc4c(c3)oc3ccccc34)cc2)cc1 ; c1ccc(-c2ccc(N(c3ccc(-c4ccccc4)cc3)c3cc(-c4ccccc4)c(-c4ccccc4)cc3N(c3ccc(-c4ccccc4)cc3)c3ccc4c(c3)oc3ccccc34)cc2)cc1 ; c1ccc(-c2ccc(N(c3cc(-c4ccccc4)c(-c4ccccc4)cc3N(c3ccc(-c4ccccc4)cc3)c3cccc4c3oc3ccccc34)c3cccc4c3oc3ccccc34)cc2)cc1]

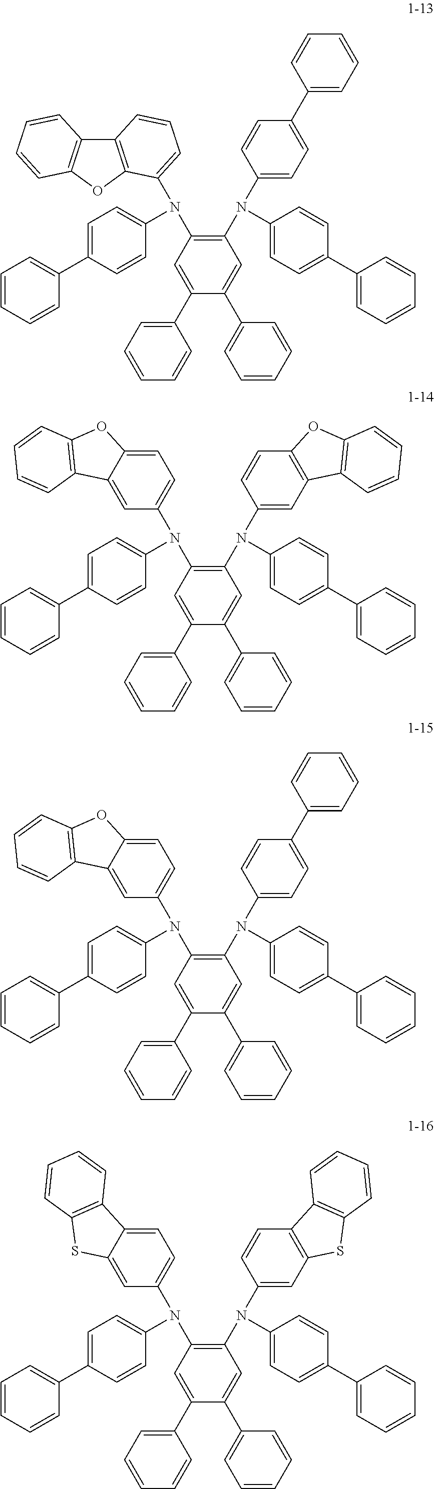 [SMILES: c1ccc(-c2ccc(N(c3ccc(-c4ccccc4)cc3)c3cc(-c4ccccc4)c(-c4ccccc4)cc3N(c3ccc(-c4ccccc4)cc3)c3cccc4c3oc3ccccc34)cc2)cc1 ; c1ccc(-c2ccc(N(c3ccc4oc5ccccc5c4c3)c3cc(-c4ccccc4)c(-c4ccccc4)cc3N(c3ccc(-c4ccccc4)cc3)c3ccc4oc5ccccc5c4c3)cc2)cc1 ; c1ccc(-c2ccc(N(c3ccc(-c4ccccc4)cc3)c3cc(-c4ccccc4)c(-c4ccccc4)cc3N(c3ccc(-c4ccccc4)cc3)c3ccc4oc5ccccc5c4c3)cc2)cc1 ; c1ccc(-c2ccc(N(c3ccc4c(c3)sc3ccccc34)c3cc(-c4ccccc4)c(-c4ccccc4)cc3N(c3ccc(-c4ccccc4)cc3)c3ccc4c(c3)sc3ccccc34)cc2)cc1]

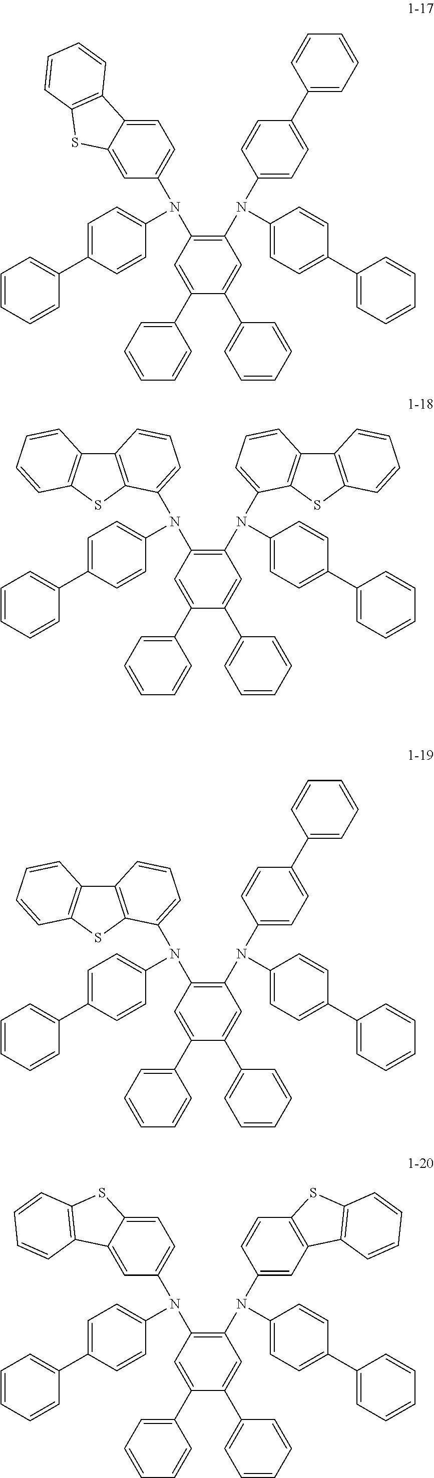 [SMILES: c1ccc(-c2ccc(N(c3ccc(-c4ccccc4)cc3)c3cc(-c4ccccc4)c(-c4ccccc4)cc3N(c3ccc(-c4ccccc4)cc3)c3ccc4c(c3)sc3ccccc34)cc2)cc1 ; c1ccc(-c2ccc(N(c3cc(-c4ccccc4)c(-c4ccccc4)cc3N(c3ccc(-c4ccccc4)cc3)c3cccc4c3sc3ccccc34)c3cccc4c3sc3ccccc34)cc2)cc1 ; c1ccc(-c2ccc(N(c3ccc(-c4ccccc4)cc3)c3cc(-c4ccccc4)c(-c4ccccc4)cc3N(c3ccc(-c4ccccc4)cc3)c3cccc4c3sc3ccccc34)cc2)cc1 ; c1ccc(-c2ccc(N(c3ccc4sc5ccccc5c4c3)c3cc(-c4ccccc4)c(-c4ccccc4)cc3N(c3ccc(-c4ccccc4)cc3)c3ccc4sc5ccccc5c4c3)cc2)cc1]

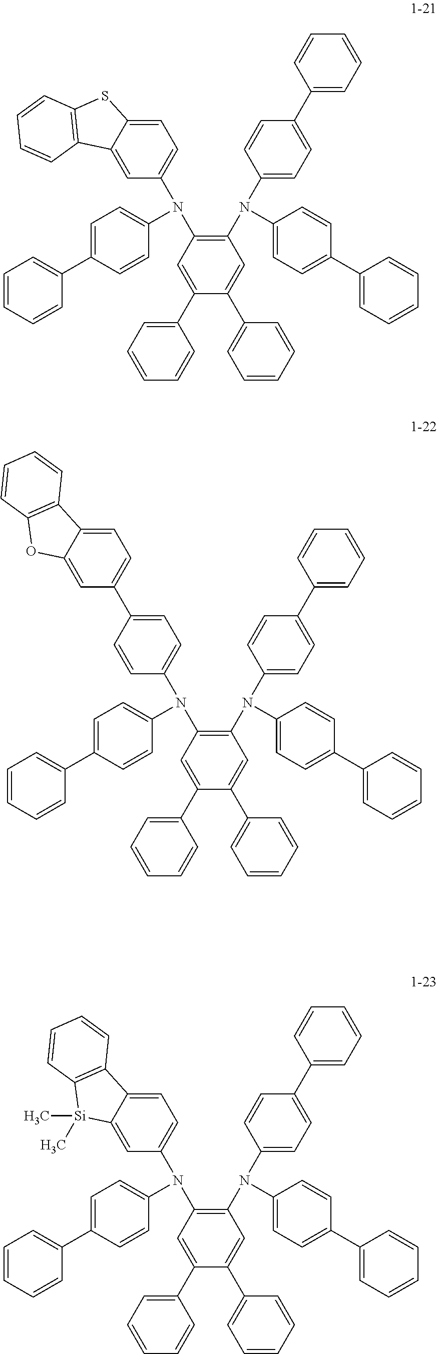 [SMILES: c1ccc(-c2ccc(N(c3ccc(-c4ccccc4)cc3)c3cc(-c4ccccc4)c(-c4ccccc4)cc3N(c3ccc(-c4ccccc4)cc3)c3ccc4sc5ccccc5c4c3)cc2)cc1 ; c1ccc(-c2ccc(N(c3ccc(-c4ccccc4)cc3)c3cc(-c4ccccc4)c(-c4ccccc4)cc3N(c3ccc(-c4ccccc4)cc3)c3ccc(-c4ccc5c(c4)oc4ccccc45)cc3)cc2)cc1 ; C[Si]1(C)c2ccccc2-c2ccc(N(c3ccc(-c4ccccc4)cc3)c3cc(-c4ccccc4)c(-c4ccccc4)cc3N(c3ccc(-c4ccccc4)cc3)c3ccc(-c4ccccc4)cc3)cc21]

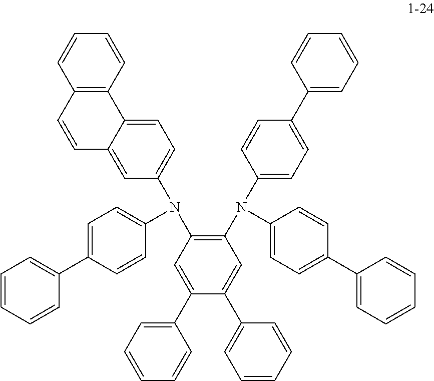 [SMILES: c1ccc(-c2ccc(N(c3ccc(-c4ccccc4)cc3)c3cc(-c4ccccc4)c(-c4ccccc4)cc3N(c3ccc(-c4ccccc4)cc3)c3ccc4c(ccc5ccccc54)c3)cc2)cc1]

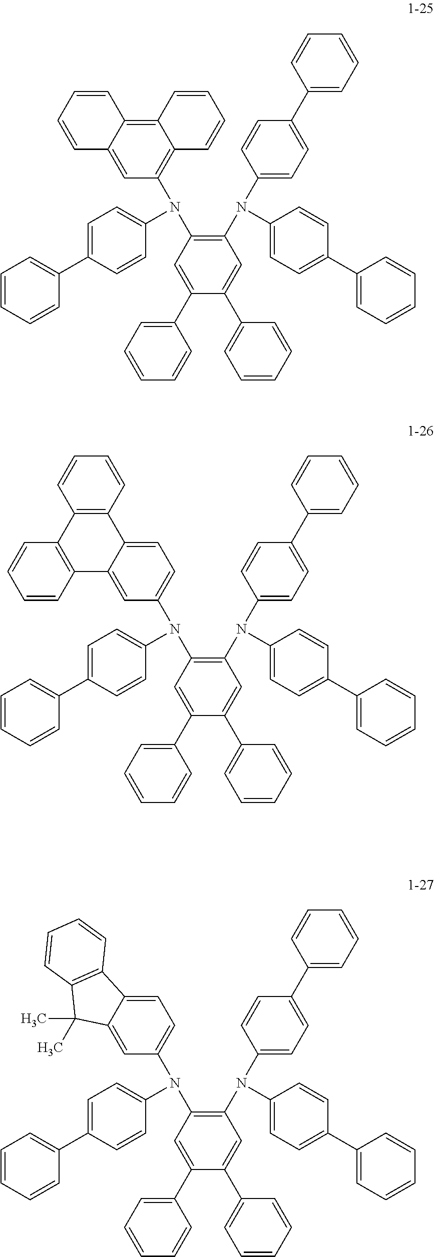 [SMILES: c1ccc(-c2ccc(N(c3ccc(-c4ccccc4)cc3)c3cc(-c4ccccc4)c(-c4ccccc4)cc3N(c3ccc(-c4ccccc4)cc3)c3cc4ccccc4c4ccccc34)cc2)cc1 ; c1ccc(-c2ccc(N(c3ccc(-c4ccccc4)cc3)c3cc(-c4ccccc4)c(-c4ccccc4)cc3N(c3ccc(-c4ccccc4)cc3)c3ccc4c5ccccc5c5ccccc5c4c3)cc2)cc1 ; CC1(C)c2ccccc2-c2ccc(N(c3ccc(-c4ccccc4)cc3)c3cc(-c4ccccc4)c(-c4ccccc4)cc3N(c3ccc(-c4ccccc4)cc3)c3ccc(-c4ccccc4)cc3)cc21]

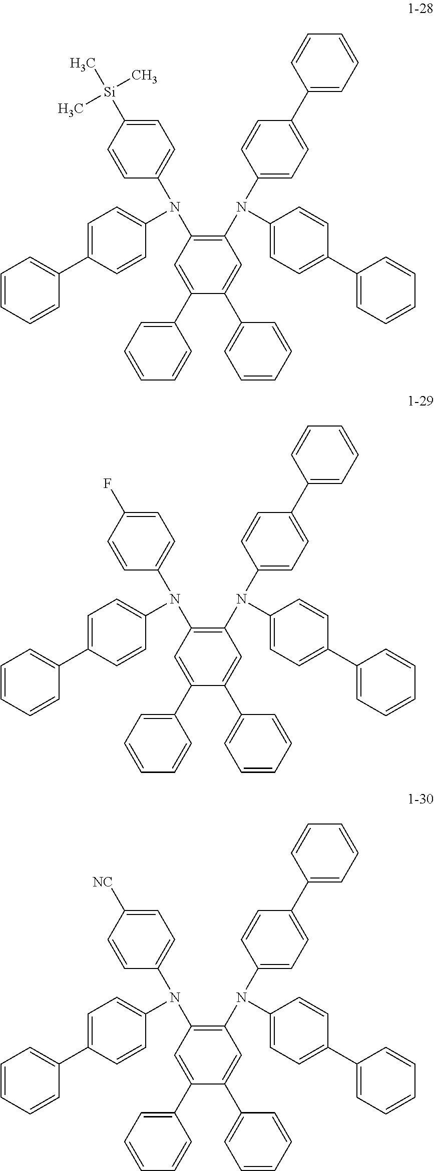 [SMILES: C[Si](C)(C)c1ccc(N(c2ccc(-c3ccccc3)cc2)c2cc(-c3ccccc3)c(-c3ccccc3)cc2N(c2ccc(-c3ccccc3)cc2)c2ccc(-c3ccccc3)cc2)cc1 ; Fc1ccc(N(c2ccc(-c3ccccc3)cc2)c2cc(-c3ccccc3)c(-c3ccccc3)cc2N(c2ccc(-c3ccccc3)cc2)c2ccc(-c3ccccc3)cc2)cc1 ; N#Cc1ccc(N(c2ccc(-c3ccccc3)cc2)c2cc(-c3ccccc3)c(-c3ccccc3)cc2N(c2ccc(-c3ccccc3)cc2)c2ccc(-c3ccccc3)cc2)cc1]

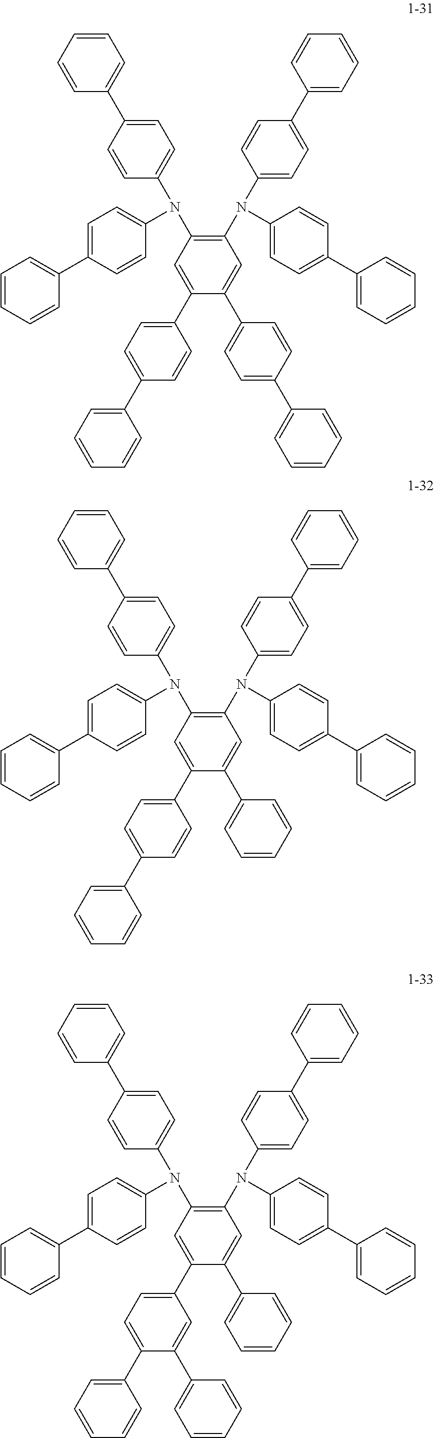 [SMILES: c1ccc(-c2ccc(-c3cc(N(c4ccc(-c5ccccc5)cc4)c4ccc(-c5ccccc5)cc4)c(N(c4ccc(-c5ccccc5)cc4)c4ccc(-c5ccccc5)cc4)cc3-c3ccc(-c4ccccc4)cc3)cc2)cc1 ; c1ccc(-c2ccc(-c3cc(N(c4ccc(-c5ccccc5)cc4)c4ccc(-c5ccccc5)cc4)c(N(c4ccc(-c5ccccc5)cc4)c4ccc(-c5ccccc5)cc4)cc3-c3ccccc3)cc2)cc1 ; c1ccc(-c2ccc(N(c3ccc(-c4ccccc4)cc3)c3cc(-c4ccccc4)c(-c4ccc(-c5ccccc5)c(-c5ccccc5)c4)cc3N(c3ccc(-c4ccccc4)cc3)c3ccc(-c4ccccc4)cc3)cc2)cc1]

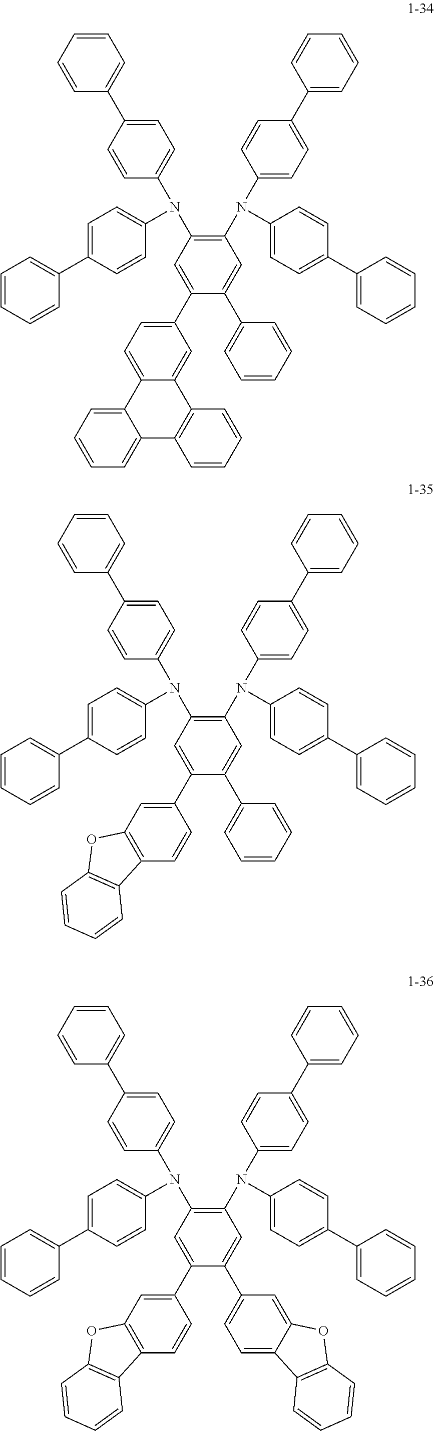 [SMILES: c1ccc(-c2ccc(N(c3ccc(-c4ccccc4)cc3)c3cc(-c4ccccc4)c(-c4ccc5c6ccccc6c6ccccc6c5c4)cc3N(c3ccc(-c4ccccc4)cc3)c3ccc(-c4ccccc4)cc3)cc2)cc1 ; c1ccc(-c2ccc(N(c3ccc(-c4ccccc4)cc3)c3cc(-c4ccccc4)c(-c4ccc5c(c4)oc4ccccc45)cc3N(c3ccc(-c4ccccc4)cc3)c3ccc(-c4ccccc4)cc3)cc2)cc1 ; c1ccc(-c2ccc(N(c3ccc(-c4ccccc4)cc3)c3cc(-c4ccc5c(c4)oc4ccccc45)c(-c4ccc5c(c4)oc4ccccc45)cc3N(c3ccc(-c4ccccc4)cc3)c3ccc(-c4ccccc4)cc3)cc2)cc1]

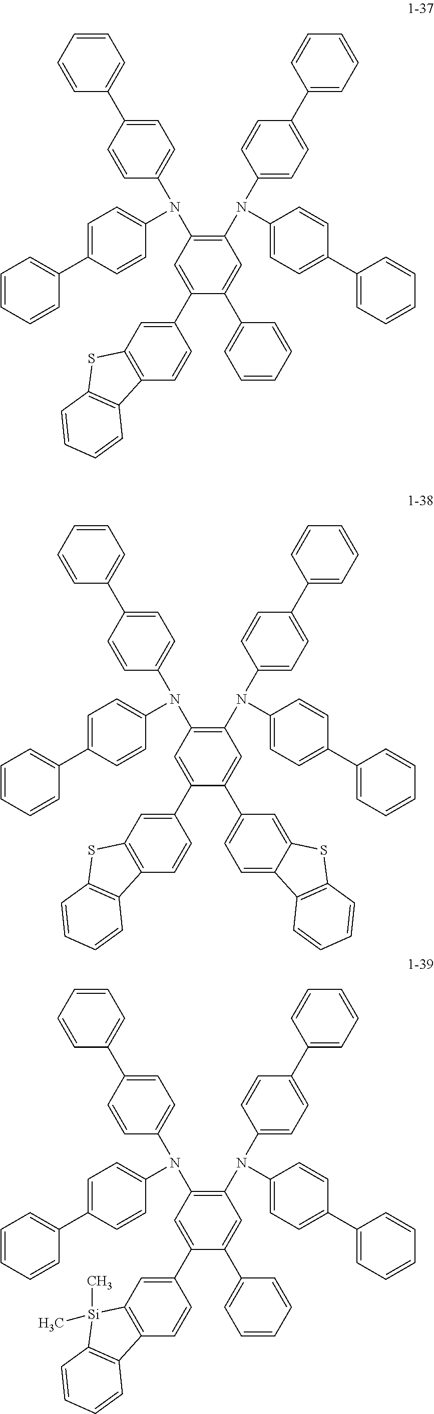

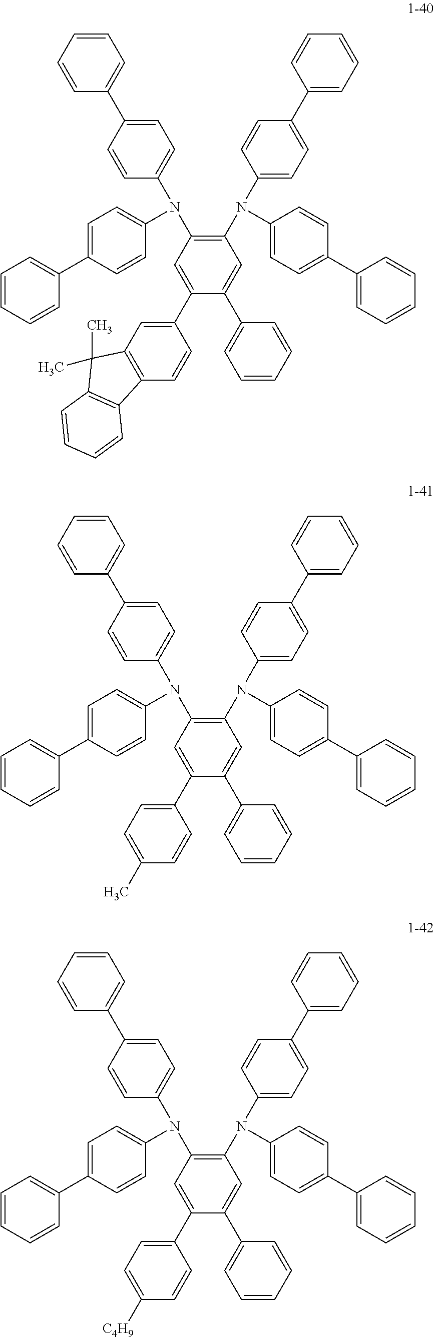

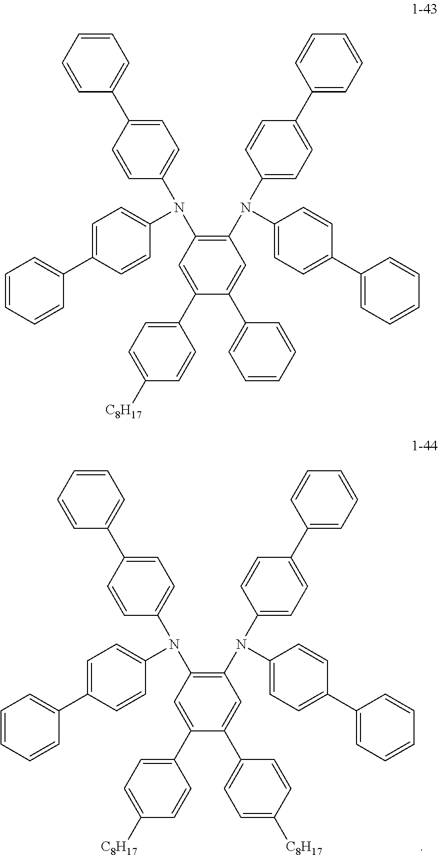

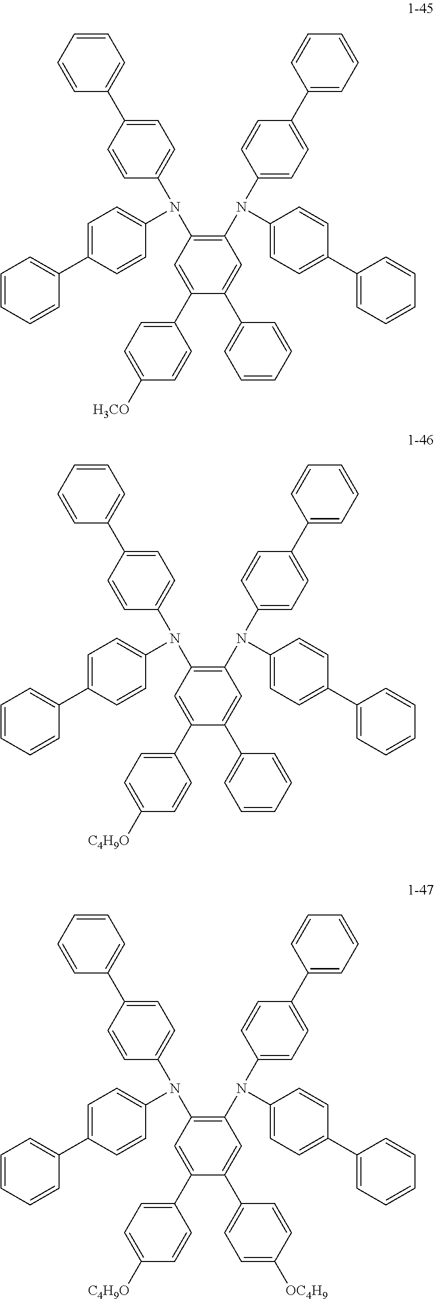

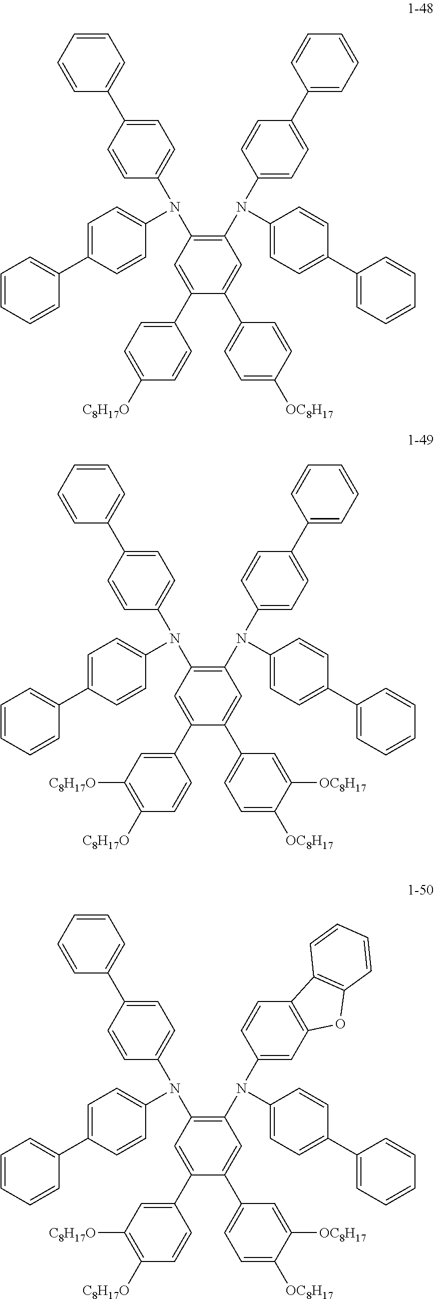

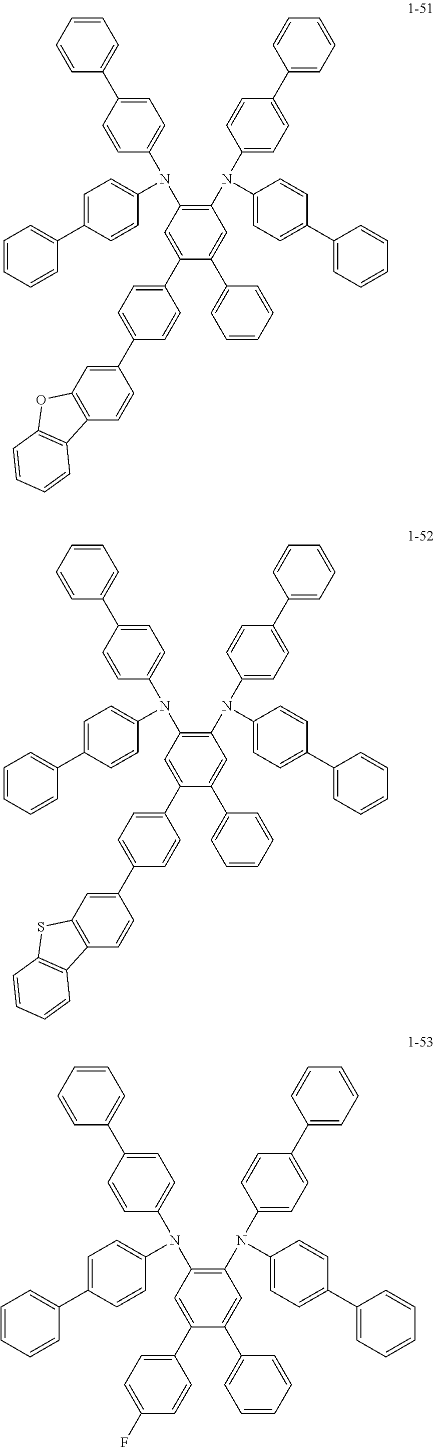 [SMILES: c1ccc(-c2ccc(N(c3ccc(-c4ccccc4)cc3)c3cc(-c4ccccc4)c(-c4ccc(-c5ccc6c(c5)oc5ccccc56)cc4)cc3N(c3ccc(-c4ccccc4)cc3)c3ccc(-c4ccccc4)cc3)cc2)cc1 ; c1ccc(-c2ccc(N(c3ccc(-c4ccccc4)cc3)c3cc(-c4ccccc4)c(-c4ccc(-c5ccc6c(c5)sc5ccccc56)cc4)cc3N(c3ccc(-c4ccccc4)cc3)c3ccc(-c4ccccc4)cc3)cc2)cc1 ; Fc1ccc(-c2cc(N(c3ccc(-c4ccccc4)cc3)c3ccc(-c4ccccc4)cc3)c(N(c3ccc(-c4ccccc4)cc3)c3ccc(-c4ccccc4)cc3)cc2-c2ccccc2)cc1]

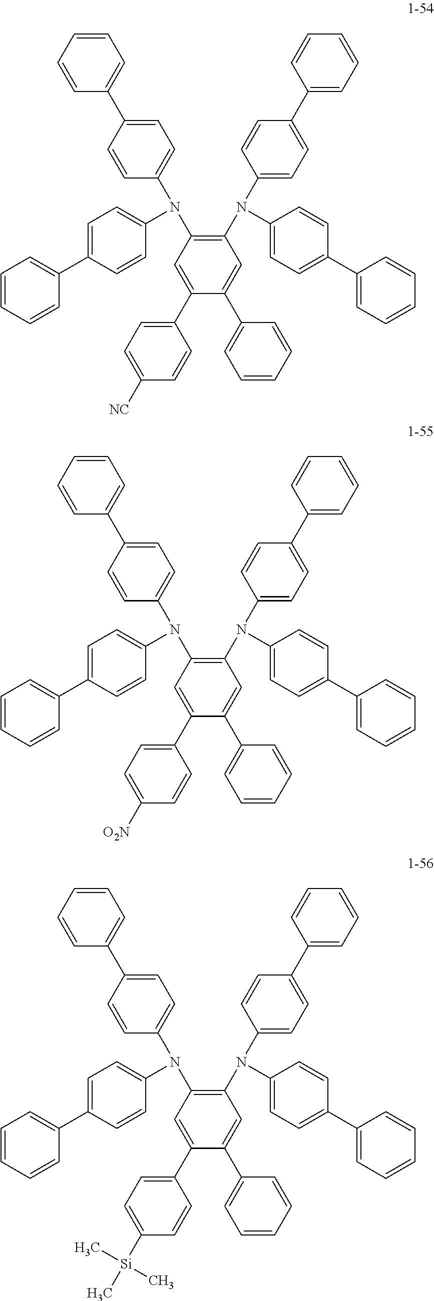 [SMILES: N#Cc1ccc(-c2cc(N(c3ccc(-c4ccccc4)cc3)c3ccc(-c4ccccc4)cc3)c(N(c3ccc(-c4ccccc4)cc3)c3ccc(-c4ccccc4)cc3)cc2-c2ccccc2)cc1 ; O=[N+]([O-])c1ccc(-c2cc(N(c3ccc(-c4ccccc4)cc3)c3ccc(-c4ccccc4)cc3)c(N(c3ccc(-c4ccccc4)cc3)c3ccc(-c4ccccc4)cc3)cc2-c2ccccc2)cc1 ; C[Si](C)(C)c1ccc(-c2cc(N(c3ccc(-c4ccccc4)cc3)c3ccc(-c4ccccc4)cc3)c(N(c3ccc(-c4ccccc4)cc3)c3ccc(-c4ccccc4)cc3)cc2-c2ccccc2)cc1]

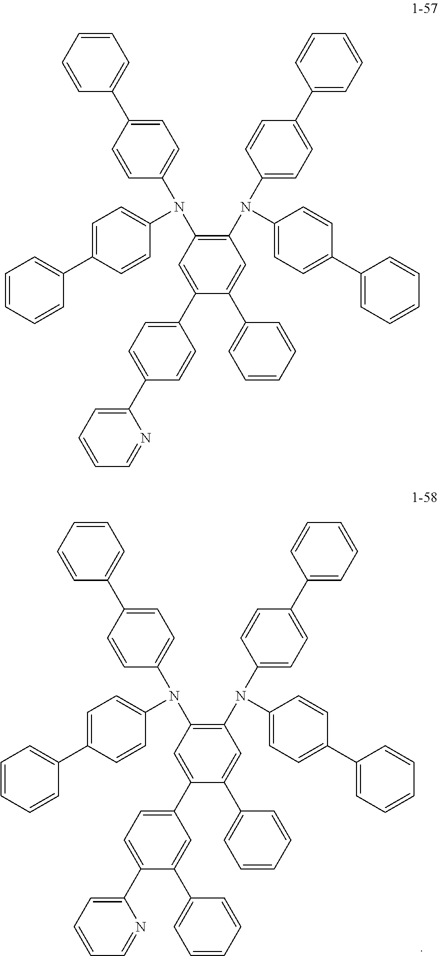 [SMILES: c1ccc(-c2ccc(N(c3ccc(-c4ccccc4)cc3)c3cc(-c4ccccc4)c(-c4ccc(-c5ccccn5)cc4)cc3N(c3ccc(-c4ccccc4)cc3)c3ccc(-c4ccccc4)cc3)cc2)cc1 ; c1ccc(-c2ccc(N(c3ccc(-c4ccccc4)cc3)c3cc(-c4ccccc4)c(-c4ccc(-c5ccccn5)c(-c5ccccc5)c4)cc3N(c3ccc(-c4ccccc4)cc3)c3ccc(-c4ccccc4)cc3)cc2)cc1]

<2. Organic Light-Emitting Device Including Compound for Organic Electroluminescence Device>

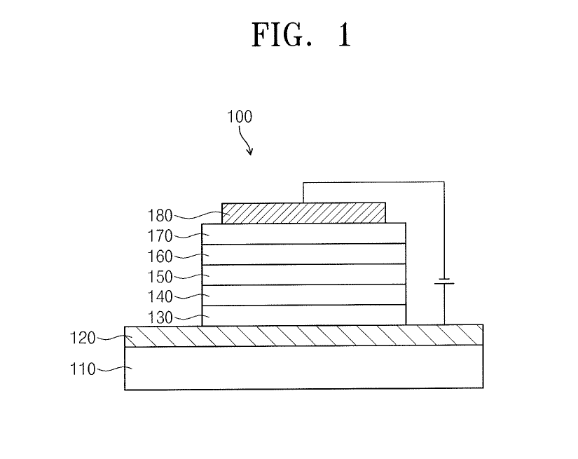

Next, the organic light-emitting device including the material for an organic electroluminescence device according to the present embodiment will be described in brief with reference to FIG. 1. FIG. 1 illustrates a schematic cross-sectional view of an example of the organic light-emitting device according to the embodiment.

As illustrated in FIG. 1, an organic light-emitting device 100 according to the embodiment may include, e.g., a substrate 110, a first electrode 120 disposed on the substrate 110, a hole injection layer 130 disposed on the first electrode 120, a hole transport layer 140 disposed on the hole injection layer 130, an emission layer 150 disposed on the hole transport layer 140, an electron transport layer 160 disposed on the emission layer 150, an electron injection layer 170 disposed on the electron transport layer 160, and a second electrode 180 disposed on the electron injection layer 170.

The compound for an organic electroluminescence device according to the present embodiment may be a material included in any one of the hole transport layer and the emission layer. In an implementation, the compound for an organic electroluminescence device may be included in both of the layers, e.g., may be included in both the hole transport layer and the emission layer. In an implementation, the compound for an organic electroluminescence device may be included in the hole transport layer 140.

Each organic thin film layer between the first electrode 120 and the second electrode 180 of the organic light-emitting device may be formed vis various suitable methods, e.g., a deposition method or the like.

A substrate for an organic light-emitting device may be used as the substrate 110. For example, the substrate 110 may be a glass substrate, a semiconductor substrate, a transparent plastic substrate, or the like.

The first electrode 120 may be, e.g., a positive electrode, and may be formed on the substrate 110 by using a deposition method, a sputtering method, or the like. For example, the first electrode 120 may be formed as a transmissive electrode by metal, an alloy, a conductive compound, and the like having a large work function. The first electrode 120 may be formed using, e.g., indium tin oxide (ITO), indium zinc oxide (IZO), tin oxide (SnO₂), zinc oxide (ZnO), or the like, which is transparent and has excellent conductivity. In an implementation, the positive electrode 120 may be formed as a reflective electrode by using magnesium (Mg), aluminum (Al), or the like.

The hole injection layer 130 is a layer for facilitating injection of holes from the first electrode 120, and, e.g., may formed to a thickness of about 10 nm to about 150 nm on the first electrode 120. The hole injection layer 130 may be formed by using a suitable material. Examples of the suitable material may include triphenylamine-containing polyether ketone (TPAPEK), 4-isopropyl-4′-methyldiphenyliodoniumtetrakis(pentafluorophenyl) boric acid (PPBI), N,N-diphenyl-N,N′-bis-[4-(phenyl-m-tolyl-amino)-phenyl]-biphenyl-4,4′-diamine (DNTPD), a phthalocyanine compound such as copper phthalocyanine, 4,4′,4″-tris(3-methylphenylamino)triphenylamine (m-MTDATA), N,N′-di(1-naphthyl)-N,N′-diphenylbenzidine (NPB), 4,4′,4″-tris{N,N diamino}triphenylamine (TDATA), 4,4′,4″-tris(N,N-2-naphthylphenylamino)triphenylamine (2-TNATA), polyaniline/dodecylbenzenesulfonic acid (Pani/DBSA), poly(3,4-ethylenedioxythiophene)/poly(4-styrene sulfonate) (PEDOT/PSS), polyaniline/camphorsulfonic acid (Pani/CSA), polyaniline/poly(4-styrene sulfonate) (PANI/PSS), or the like.

The hole transport layer 140 may include a hole transport material having a function of transporting holes, and, e.g., may be formed to a thickness of about 10 nm to about 150 nm on the hole injection layer 130. In an implementation, the hole transport layer 140 may be formed of or include the compound for an organic electroluminescence device according to the present embodiment. In an implementation, in the case where the compound for an organic electroluminescence device according to the present embodiment is used as a host material of the emission layer 150, the hole transport layer 140 may be formed by using a suitable hole transport material. Examples of the suitable hole transport material may include 1,1-bis[(di-4-tolylamino)phenyl]cyclohexane (TAPC), a carbazole derivative such as N-phenyl carbazole and polyvinyl carbazole, N,N′-bis(3-methylphenyl)-N,N′-diphenyl-[1,1-biphenyl]-4,4′-diamine (TPD), 4,4′,4″-tris(N-carbazolyl)triphenylamine (TCTA), N,N′-di(1-naphthyl)-N,N′-diphenylbenzidine (NPB), and the like.

The emission layer 150 may emit light by fluorescence, phosphorescence, or the like. The emission layer 150 may include a host material and a dopant material as an emission material. In an implementation, the emission layer 150 may be formed to a thickness of about 10 nm to about 60 nm.

The compound for an organic electroluminescence device according to the present embodiment may be used or included as the host material of the emission layer 150. In an implementation, in the case where the compound for an organic electroluminescence device according to the present embodiment is used as the hole transport material, the host material of the emission layer 150 may be a suitable host material.

The host material of the emission layer 150 may include, e.g., tris(8-quinolinolato)aluminum (Alq₃), 4,4′-N,N′-dicarbazole-biphenyl (CBP), poly(n-vinylcarbazole) (PVK), 9,10-di(naphthalene-2-yl)anthracene (ADN), 4,4′,4″-tris(N-carbazolyl)triphenylamine (TCTA), 1,3,5-tris(N-phenylbenzimidazole-2-yl)benzene (TPBI), 3-tert-butyl-9,10-di(naphtho-2-yl)anthracene (TBADN), distyrylarylene (DSA), 4,4′-bis(9-carbazole)-2,2′-dimethyl-biphenyl (dmCBP), or the like.

The emission layer 150 may be formed as an emission layer for emitting light having a predetermined color. For example, the emission layer 150 may be formed as a red emission layer, a green emission layer, or a blue emission layer.

In the case where the emission layer 150 is the blue emission layer, a suitable material may be used as a blue dopant, e.g., perylene and a derivative thereof, an iridium (Ir) complex such as bis[2-(4,6-difluorophenyl)pyridinate]picolinate iridium (III) (FIrpic), or the like may be used.

In the case where the emission layer 150 is the red emission layer, a suitable material may be used as a red dopant, e.g., rubrene and a derivative thereof, 4-dicyanomethylene-2-(p-dimethylaminostyryl)-6-methyl-4H-pyran (DCM) and a derivative thereof, an iridium complex such as bis(1-phenylisoquinoline)(acetylacetonate)iridium (III) (Ir(piq)₂(acac)), an osmium (Os) complex, a platinum complex, or the like may be used.

In the case where the emission layer 150 is the green emission layer, a suitable material may be used as a green dopant, e.g., coumarin and a derivative thereof, an iridium complex such as tris(2-phenylpyridine)iridium (III) (Ir(ppy)₃), or the like may be used.

The electron transport layer 160 may include an electron transport material having a function of transporting electrons, and, e.g., may be formed to a thickness of about 15 nm to about 50 nm on the emission layer 150. The electron transport layer 160 may be formed by using a suitable electron transport material. Examples of the suitable electron transport material may include a quinoline derivative such as tris(8-quinolinolato)aluminum (Alq₃), 1,2,4-triazole derivative (TAZ), bis(2-methyl-8-quinolinolato)-(p-phenylphenolate)-aluminum (BAlq), beryllium bis(benzoquinoline-10-olate) (BeBq₂), a Li complex such as lithium quinolate (LiQ), and the like.

The electron injection layer 170 may facilitate injection of electrons from the second electrode 180, and may be formed to a thickness of about 0.3 nm to about 9 nm. In an implementation, the electron injection layer 170 may be formed by using a suitable material, e.g., lithium fluoride (LiF), sodium chloride (NaCl), cesium fluoride (CsF), lithium oxide (Li₂O), barium oxide (BaO), or the like.

The second electrode 180 may be, e.g., a negative electrode. For example, the second electrode 116 is formed as a reflective electrode by metal, an alloy, a conductive compound, and the like having a small work function. The second electrode 180 may be formed of, e.g., lithium (Li), magnesium (Mg), aluminum (Al), aluminum-lithium (Al—Li), calcium (Ca), magnesium-indium (Mg—In), magnesium-silver (Mg—Ag), or the like. In an implementation, the second electrode 180 may be formed as a transmissive electrode by using indium tin oxide (ITO), indium zinc oxide (IZO), or the like. The second electrode 180 may be formed on the electron injection layer 170, e.g., by using a deposition method, a sputtering method, or the like.

An example of the structure of the organic light-emitting device 100 according to the present embodiment is described above. In the organic light-emitting device 100 including the compound for an organic electroluminescence device according to the present embodiment, the HOMO level of the compound for an organic electroluminescence device may be low and a stable cationic radical may be formed, and hole transport properties may be improved. Accordingly, the driving voltage, emission efficiency, and the emission life of the organic electroluminescence device may be improved.

In an implementation, the organic light-emitting device 100 according to the present embodiment may be formed by using various typical other structures of the organic light-emitting device. For example, the organic light-emitting device 100 may not include one or more layers of the hole injection layer 130, the electron transport layer 160, and the electron injection layer 170. Further, each layer of the organic light-emitting device 100 may be formed of a single layer or a plurality of layers.

In an implementation, the organic light-emitting device 100 may include a hole blocking layer between the hole transport layer 140 and the emission layer 150 to help prevent diffusion of triplet excitons or holes into the electron transport layer 160. In an implementation, the hole blocking layer may be formed using, e.g., an oxadiazole derivative, a triazole derivative, a phenanthroline derivative, or the like.

### Example

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Further, in the embodiment, compounds represented by Chemical Formulas 1-1 to 1-58 (examples of materials for an organic electroluminescence device according to the embodiment) are also called Compounds 1-1 to 1-58.

### Synthetic Example 1

### Synthesis of Compound 1-2

Compound 1-2 was synthesized according to the following synthesis scheme.

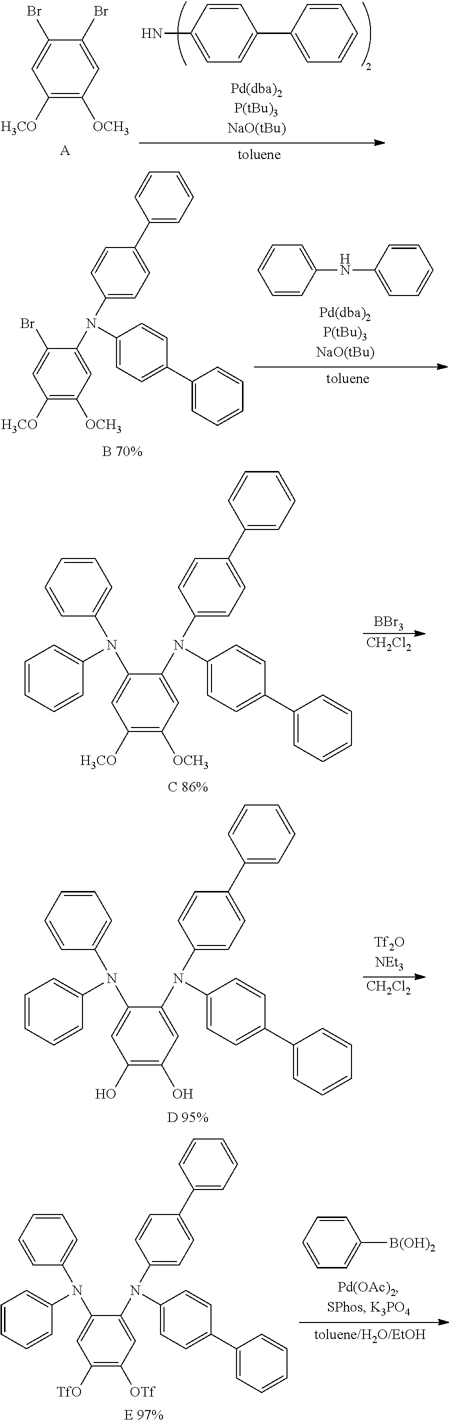

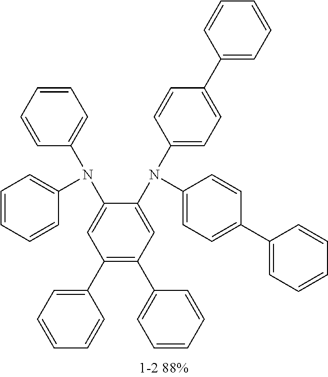 [SMILES: c1ccc(-c2ccc(N(c3ccc(-c4ccccc4)cc3)c3cc(-c4ccccc4)c(-c4ccccc4)cc3N(c3ccccc3)c3ccccc3)cc2)cc1]

### Synthesis of Compound B

Under an argon atmosphere, about 10.00 g of Compound A, about 2.17 g of bis(4-biphenylyl)amine, about 0.19 g of bis(dibenzylideneacetone)palladium(0), about 0.27 g of tri-tert-butylphosphine, and about 0.97 g of sodium tert-butoxide were added to a 500 mL three-neck flask, and heated and refluxed in about 150 mL of toluene solvent for about 4 hours. After air cooling, water was added to the reaction solution to separate an organic layer and solvent was removed by distillation. The obtained crude product was purified by silica gel column chromatography (toluene/hexane) to obtain about 2.54 g of Compound B as a white solid (yield about 70%). The molecular weight of obtained Compound B was measured by FAB-MS to obtain a value of 536(C₃₂H₂₆BrNO₂).

### Synthesis of Compound C

Under an argon atmosphere, about 2.54 g of Compound B, about 0.80 g of diphenylamine, about 0.14 g of bis(dibenzylideneacetone)palladium(0), about 0.19 g of tri-tert-butylphosphine, and about 0.68 g of sodium tert-butoxide were added to a 200 mL three-neck flask, and heated and refluxed in about 50 mL of toluene solvent for about 4 hours. After air cooling, water was added to the reaction solution to separate an organic layer and solvent was removed by distillation. The obtained crude product was purified by silica gel column chromatography (toluene) to obtain about 2.55 g of Compound C as a white solid (yield about 86%). The molecular weight of obtained Compound C was measured by FAB-MS to obtain a value of 624 (C₄₄H₃₆N₂O₂).

### Synthesis of Compound D

Under an argon atmosphere, in a 200 mL three-neck flask, about 2.55 g of Compound C was dissolved in about 100 mL of dichloromethane, and about 10.2 mL of a dichloromethane solution of boron tribromide (about 1 mol/L) was dripped in the flask while being maintained in an ice bath. After the reaction solution was agitated at ambient temperature for about 6 hours, water was added to the reaction solution to separate an organic layer and solvent was removed by distillation. The obtained crude product was purified by silica gel column chromatography (toluene and ethyl acetate) to obtain about 2.31 g of Compound D as a lemon yellow solid (yield about 95%). The molecular weight of Compound D was measured by FAB-MS to obtain a value of 596 (C₄₂H₃₂N₂O₂).

### Synthesis of Compound E

Under an argon atmosphere, in a 200 mL three-neck flask, about 2.31 g of Compound D was dissolved in about 60 mL of dichloromethane, and while the flask was in an ice bath, about 2.7 mL of triethylamine was added, and about 1.6 mL of trifluoromethylsulfonic acid anhydride was then added dropwise. After the reaction solution was agitated at ambient temperature for about 2 hours, water was added to the reaction solution to separate the organic layer and solvent was removed by distillation. The obtained crude product was purified by silica gel column chromatography (dichloromethane) to obtain about 3.23 g of Compound E as a lemon yellow solid (yield about 97%). The molecular weight of Compound E was measured by FAB-MS to obtain a value of 860 (C₄₄H₃₀F₆N₂O₆S₂).

### Synthesis of Compound 1-2

Under an argon atmosphere, in a 200 mL three-neck flask, about 3.23 g of compound E, about 1.14 g of phenyl boronic acid, about 0.08 g of palladium acetate, about 0.31 g of 2-dicyclohexylphosphino-2′,6′-dimethoxybiphenyl, and about 3.19 g of tripotassium phosphate were agitated in about 50 mL of a mixed solvent of toluene and water/ethanol (about 10:1:1 (volume ratio)) at about 100° C. for about 16 hours. Water was added to the reaction solution to separate the organic layer and solvent was removed by distillation. The obtained crude product was purified by silica gel column chromatography (toluene and hexane) to obtain about 2.37 g of Compound 1-2 as a white solid (yield about 88%). The molecular weight of Compound 1-2 was measured by FAB-MS to obtain a value of 716 (C₅₄H₄₀N₂). ¹H NMR (300 MHz, DMSO-d₆) of Compound 1-2 was measured to obtain the following chemical shifts. 7.66-7.32 (m, 30H), 7.15 (s, 1H), 7.12 (s, 1H), 6.99 (d, J=8.5 Hz, 4H), 6.93 (d, J=8.3 Hz, 4H). Accordingly, it could be identified that Compound 1-2 was synthesized.

### Synthetic Example 2

### Synthesis of Compound 1-5

Compound 1-5 was synthesized according to the following synthesis scheme.

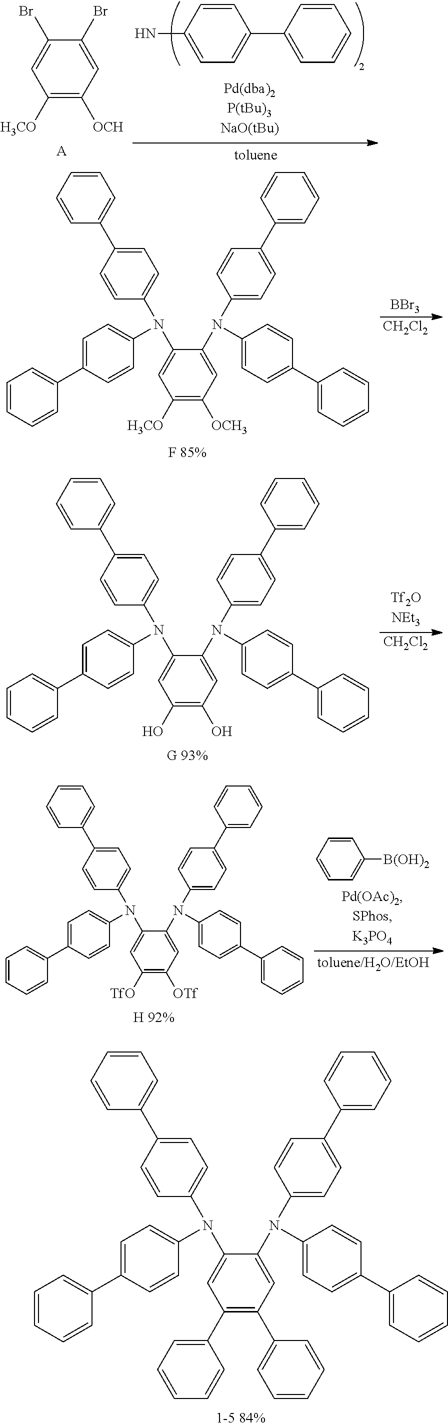

### Synthesis of Compound F

Under an argon atmosphere, about 1.50 g of Compound A, about 3.26 g of bis(4-biphenylyl)amine, about 0.15 g of bis(dibenzylideneacetone)palladium(0), about 0.20 g of tri-tert-butylphosphine, and about 1.22 g of sodium tert-butoxide were added to a 200 mL three-neck flask, and heated and refluxed in about 50 mL of toluene solvent for about 20 hours. After air cooling, water was added to the reaction solution to separate an organic layer and solvent was removed by distillation. The obtained crude product was purified by silica gel column chromatography (toluene) to obtain about 3.34 g of Compound F as a white solid (yield about 85%). The molecular weight of Compound F was measured by FAB-MS to obtain a value of 776(C₅₅H₄₄N₂O₂).

### Synthesis of Compound G

Under an argon atmosphere, in a 200 mL three-neck flask, about 3.34 g of Compound F was dissolved in about 100 mL of dichloromethane, and about 10.7 mL of a dichloromethane solution of boron tribromide (about 1 mol/L) was added dropwise to the flask as the flask was held in the ice bath. After the reaction solution was agitated at ambient temperature for about 6 hours, water was added to the reaction solution to separate the organic layer and solvent was removed by distillation. The obtained crude product was purified by silica gel column chromatography (toluene and ethyl acetate) to obtain about 2.99 g of Compound G as a lemon yellow solid (yield about 93%). The molecular weight of Compound G was measured by FAB-MS to obtain a value of 748(C₅₄H₄₀N₂O₂).

### Synthesis of Compound H

Under an argon atmosphere, in a 200 mL three-neck flask, about 2.99 g of Compound G was dissolved in about 80 mL of dichloromethane, and as the flask was held in an ice bath, about 2.8 mL of triethylamine was added, and about 1.7 mL of trifluoromethylsulfonic acid anhydride was added dropwise. After the reaction solution was agitated at ambient temperature for about 2 hours, water was added to the reaction solution to separate the organic layer and solvent was removed by distillation. The obtained crude product was purified by silica gel column chromatography (dichloromethane) to obtain about 3.72 g of Compound H as a lemon yellow solid (yield about 92%). The molecular weight of Compound H was measured by FAB-MS to obtain a value of 1013 (C₅₆H₃₈F₆N₂O₆S₂).

### Synthesis of Compound 1-5

Under an argon atmosphere, in a 200 mL three-neck flask, about 3.72 g of compound H, about 1.12 g of phenyl boronic acid, about 0.08 g of palladium acetate, about 0.30 g of 2-dicyclohexylphosphino-2′,6′-dimethoxybiphenyl, and about 3.11 g of tripotassium phosphate were agitated in about 50 mL of mixed solvent of toluene and water/ethanol (about 10:1:1 (volume ratio)) at about 100° C. for about 6 hours. After air cooling, water was added to separate the organic layer and solvent was removed by distillation. The obtained crude product was purified by silica gel column chromatography (toluene and hexane) to obtain about 2.68 g of Compound 1-5 as a white solid (yield about 84%). The molecular weight of Compound 1-5 was measured by FAB-MS to obtain a value of 869(C₆₆H₄₈N₂). Further, ¹H NMR (300 MHz, DMSO-d₆) of Compound 1-5 was measured to obtain the following chemical shifts. 7.65-7.35 (m, 38H), 7.15 (s, 2H), 6.95 (d, J=8.5 Hz, 8H).

### Synthetic Example 3

### Synthesis of Compound 1-7

In the synthesis scheme of Compound 1-5, N-phenyl-1-napthylamine was used instead of bis(4-biphenylyl)amine to synthesize Compound 1-7. Further, the molecular weight of Compound 1-7 was measured by FAB-MS to obtain a value of 664(C₅₀H₃₆N₂). Further, ¹H NMR (300 MHz, DMSO-d₆) of Compound 1-7 was measured to obtain the chemical shifts expected from the structure of Compound 1-7. Accordingly, it could be identified that Compound 1-7 was synthesized.

### Synthetic Example 4

### Synthesis of Compound 1-10

In the synthesis scheme of Compound 1-5, 3-(biphenylamino)dibenzofuran was used instead of bis(4-biphenylyl)amine to synthesize Compound 1-10. Further, the molecular weight of Compound 1-10 was measured by FAB-MS to obtain a value of 896(C₆₆H₄₄N₂O₂). Further, ¹H NMR (300 MHz, DMSO-d₆) of Compound 1-10 was measured to obtain the chemical shifts expected from the structure of Compound 1-10. Accordingly, it could be identified that Compound 1-10 was synthesized.

### Synthetic Example 5

### Synthesis of Compound 1-16

In the synthesis scheme of Compound 1-5, 3-(biphenylylamino)dibenzothiophene was used instead of bis(4-biphenylyl)amine to synthesize Compound 1-16. Further, the molecular weight of Compound 1-16 was measured by FAB-MS to obtain a value of 928(C₆₆H₄₄N₂S₂). Further, ¹H NMR (300 MHz, DMSO-d₆) of Compound 1-16 was measured to obtain the chemical shifts expected from the structure of Compound 1-16. Accordingly, it could be identified that Compound 1-16 was synthesized.

### Synthetic Example 6

### Synthesis of Compound 1-36

In the synthesis scheme of Compound 1-5, 3-bromodibenzofuran was used instead of phenylboronic acid to synthesize Compound 1-36. In the synthesis scheme of Compound 1-5, 3-bromodibenzofuran was used instead of phenylboronic acid to synthesize Compound 1-36. Further, the molecular weight of Compound 1-36 was measured by FAB-MS to obtain a value of 1048 (C₇₈H₅₂N₂O₂). Further, ¹H NMR (300 MHz, DMSO-d₆) of Compound 1-36 was measured to obtain the chemical shifts expected from the structure of Compound 1-36. Accordingly, it could be identified that Compound 1-36 was synthesized.

(Measurement of HOMO Level)

Compounds 1-2, 1-5, 1-7, 1-10, 1-16, and 1-36 are compounds corresponding to the Examples of the present embodiment. The HOMO levels of Compounds 1-2, 1-5, 1-7, 1-10, 1-16, and 1-36 were measured by using AC-3 (photoelectron spectroscopy AC-3 in the atmosphere, manufactured by RIKEN KEIKI Co., Ltd.). Further, for comparison, the HOMO level of Comparative Compound C1 was measured. Further, Comparative Compound C1 is represented by the following Chemical Formula C1. Comparative Compound C1 is a Comparative Example. The measurement results are described in Table 1.

TABLE 1

| | HOMO (eV) |
|---|---|
| Compound 1-2 | −5.69 |
| Compound 1-5 | −5.70 |
| Compound 1-7 | −5.69 |
| Compound 1-10 | −5.74 |
| Compound 1-16 | −5.75 |
| Compound 1-36 | −5.74 |
| Comparative | −5.30 |
| Compound C1 | |

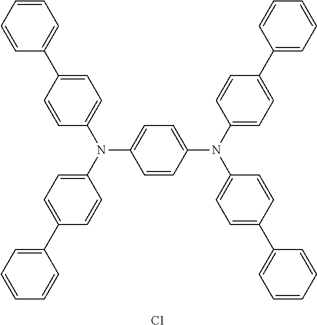 [SMILES: c1ccc(-c2ccc(N(c3ccc(-c4ccccc4)cc3)c3ccc(N(c4ccc(-c5ccccc5)cc4)c4ccc(-c5ccccc5)cc4)cc3)cc2)cc1 ; "C1"]

As may be seen in Table 1, the HOMO levels of Compounds 1-2, 1-5, 1-7, 1-10, 1-16, and 1-36 were lower than the HOMO level of Comparative Compound C1. Accordingly, it may be seen that, in the organic electroluminescence device using Compounds 1-2, 1-5, 1-7, 1-10, 1-16, and 1-36 as the hole transport material, holes may be smoothly injected from the hole injection layer to the hole transport layer.

(Measurement of Glass Transition Point)

Next, the glass transition points of Compounds 1-2, 1-5, 1-7, 1-10, 1-16, and 1-36 were measured by using a differential scanning calorimeter DSC7020 manufactured by Hitachi High-Technologies Corporation. Further, for comparison, the glass transition point of Comparative Compound C2 was measured. Further, Comparative Compound C2 is represented by the following Chemical Formula C2. Comparative Compound C2 is a Comparative Example. The measurement results are described in Table 2.

TABLE 2

| | Glass transition |
|---|---|
| | point (° C.) |
| Compound 1-2 | 116 |
| Compound 1-5 | 128 |
| Compound 1-7 | 105 |
| Compound 1-10 | 126 |
| Compound 1-16 | 132 |
| Compound 1-36 | 137 |
| Comparative | 90 |
| Compound C2 | |

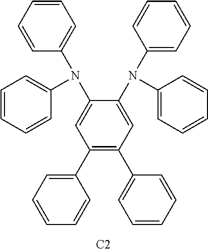

As may be seen in Table 2, the glass transition points of Compounds 1-2, 1-5, 1-7, 1-10, 1-16, and 1-36 were higher than the glass transition point of Comparative Compound C2. Accordingly, it may be seen that, in the organic electroluminescence device using Compounds 1-2, 1-5, 1-7, 1-10, 1-16, and 1-36 as the hole transport material, thermal stability of Compounds 1-2, 1-5, 1-7, 1-10, 1-16, and 1-36 may be increased and a film quality in the device may be improved.

(Manufacturing of Organic Electroluminescence Device)

Next, the organic electroluminescence device was manufactured by the following manufacturing method. First, an ITO-glass substrate patterned in advance and subjected to washing treatment was subjected to surface treatment by ozone (O₃). Further, the thickness of the ITO film (first electrode) was about 150 nm. After ozone treatment, the substrate was washed. The washed substrate was set on a glass bell jar-type deposition apparatus for forming an organic layer, and a hole injection layer, a HTL (hole transport layer), an emission layer, and an electron transport layer were sequentially deposited under a degree of vacuum of about 10⁻⁴ to 10⁻⁵ Pa. The material of the hole injection layer was 2-TNATA and the thickness was set to about 60 nm. The materials of the HTL were those described in Table 3, and the thickness was set to about 30 nm. Further, the thickness of the emission layer was set to about 25 nm. The host of the emission material was 9,10-di(2-naphthyl)anthracene (ADN). The dopant was 2,5,8,11-tetra-t-butylperylene (TBP). A doping amount of the dopant was set to about 3 mass % based on the mass of the host. The material of the electron transport layer was Alq₃ and the thickness was set to about 25 nm. Continuously, the substrate was moved by the glass bell jar-type deposition apparatus for forming the metal film, and the electron injection layer and the negative electrode material were deposited under the degree of vacuum of about 10⁻⁴ to 10⁻⁵ Pa. The material of the electron injection layer was LiF and the thickness was set to about 1.0 nm. The material of the second electrode was Al and the thickness was set to about 100 nm.

TABLE 3

| Device | | Current | Driving | Emission | |
|---|---|---|---|---|---|
| manufacturing | | density | voltage | efficiency | Emission life |
| example | HTL | (A/cm²) | (V) | (cd/A) | (LT50(hr)) |
| Example 1 | Compound 1-2 | 10 | 6.5 | 6.7 | 1,600 |
| Example 2 | Compound 1-5 | 10 | 6.3 | 7.2 | 2,100 |
| Example 3 | Compound 1-7 | 10 | 6.4 | 6.9 | 1,900 |
| Example 4 | Compound 1-10 | 10 | 6.2 | 7.4 | 1,800 |
| Example 5 | Compound 1-16 | 10 | 6.1 | 7.3 | 1,600 |
| Example 6 | Compound 1-36 | 10 | 6.3 | 7.1 | 1,900 |
| Comparative | Comparative Compound C1 | 10 | 8.1 | 5.3 | 1,100 |
| Example 1 | | | | | |
| Comparative | Comparative Compound C2 | 10 | 6.6 | 6.0 | 1,300 |
| Example 2 | | | | | |

(Property Evaluation)

Next, the driving voltage, emission efficiency, and the emission life of the manufactured organic electroluminescence device were measured. Further, in evaluation of the electroluminescence property of the manufactured organic EL device 100, the brightness orientation property measurement apparatus C9920-11 manufactured by HAMAMATSU Photonics K. K. was used. Further, in Table 3, the current density was measured to be about 10 (mA/cm²) and the half-life was measured to be about 1,000 (cd/m²). The results are described in Table 3.

According to Table 3, in Examples 1 to 6, the driving voltage was low, efficiency was high, and the life was long, compared to Comparative Examples 1 and 2. For example, in Examples 1 to 6, as compared to Comparative Example 1, the driving voltage and emission efficiency were significantly improved. The reason may be considered to be because in Examples 1 to 6, the HOMO level of the material for an organic electroluminescence device was low, and an appropriate value with respect to the organic electroluminescence device. Further, in Examples 1 to 6, as compared to Comparative Example 2, efficiency was high and the emission life was significantly improved. The reason may be considered to be because in Examples 1 to 6, the glass transition point of the material for an organic electroluminescence device was increased, stability of the molecules was improved, and the film quality was improved. Further, the reason may be considered to be because the substituent on nitrogen was substituted from the phenyl group to the biphenyl group, the dibenzohetero group, or the like, and thus stability of the cationic radical was improved. As described above, in the Examples, particularly, in the blue region, the driving voltage, emission efficiency, and the emission life of the organic electroluminescence device may be significantly improved. Further, in the Examples, the compound group may have a wide energy gap corresponding to the blue region, and the compound group can be applied even to the green to red regions.

According to an embodiment, the material for an organic electroluminescence device may have the configuration of Chemical Formula 1, and the driving voltage, emission efficiency, and the emission life of the organic electroluminescence device using the same may be significantly improved. Accordingly, the material for an organic electroluminescence device of the present embodiment may be useful for commercialization of various purposes.

By way of summation and review, a p-phenylenediamine derivative is a hole transport material that may be used in the hole transport layer, and an o-phenylenediamine derivative is an electric charge transport material that may be used in an electrophotographic photoreceptor.

In an organic electroluminescence device using a p-phenylenediamine derivative as a hole transport material, it may not be possible to obtain a satisfactory value with respect to a driving voltage, emission efficiency, and an emission life. Further, an organic electroluminescence device may be manufactured by using an o-phenylenediamine derivative as a hole transport material, but even in this organic electroluminescence device, it may not be possible to obtain a satisfactory value with respect to a driving voltage, emission efficiency, and an emission life. As described above, in the compounds, it may be impossible to obtain a satisfactory value with respect to the driving voltage, emission efficiency, and the emission life of the organic electroluminescence device.

The embodiments may provide a compound for an organic electroluminescence device that helps improve driving voltage, emission efficiency, and emission life of the organic electroluminescence device.

As described above, a compound for an organic electroluminescence device according to an embodiment may have a low HOMO level and a high glass transition point, and a driving voltage, emission efficiency, and an emission life of the organic electroluminescence device may be improved by using the compound in the organic electroluminescence device.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic electroluminescence device, the compound being represented by the following Chemical Formula 1: 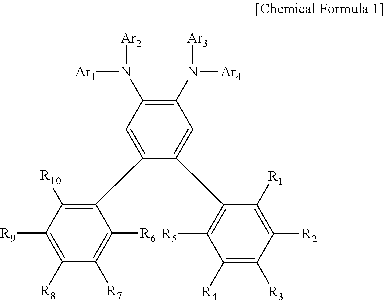
wherein, in Chemical Formula 1,
Ar₁ to Ar₄ are each independently a substituted or unsubstituted aryl group or a substituted or unsubstituted heteroaryl group,
at least one of Ar₁ to Ar₄ is a substituted or unsubstituted aryl group having 10 or more ring carbon atoms or a substituted or unsubstituted heteroaryl group having 10 or more ring carbon atoms.
R₁ to R₁₀ are each independently a hydrogen atom, a substituted or unsubstituted aryl group, a substituted or unsubstituted heteroaryl group, a substituted or unsubstituted alkyl group, a substituted or unsubstituted alkoxy group, a halogen atom, an electron suction group, or a deuterium atom, and
adjacent ones of R₁ to R₁₀ are separate or are combined to each other to form a saturated or unsaturated ring.

2. The compound for an organic electroluminescence device as claimed in claim 1, wherein the aryl group having 10 or more ring carbon atoms includes a biphenyl group, a naphthyl group, a phenanthrenyl group, or a triphenylenyl group.

3. The compound for an organic electroluminescence device as claimed in claim 2, wherein the aryl group having 10 or more ring carbon atoms includes the biphenyl group.

4. The compound for an organic electroluminescence device as claimed in claim 1, wherein the heteroaryl group having 10 or more ring carbon atoms includes a dibenzohetero group.

5. The compound for an organic electroluminescence device as claimed in claim 4, wherein the dibenzohetero group is a group represented by the following Chemical Formula 2: 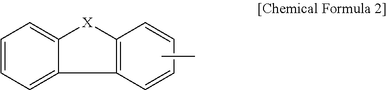
wherein, in Chemical Formula 2,
X is O, S, or Si(R₁₁)₂, and
R₁₁ is a substituted or unsubstituted alkyl group or a substituted or unsubstituted aryl group.

6. The compound for an organic electroluminescence device as claimed in claim 1, wherein Ar₁ to Ar₄ are each independently a substituted or unsubstituted aryl group having 10 or more ring carbon atoms or a substituted or unsubstituted heteroaryl group having 10 or more ring carbon atoms.

7. An organic electroluminescence device, comprising a compound for an organic electroluminescence device, the compound being represented by the following Chemical Formula 1: 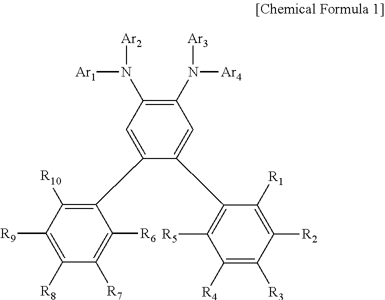
wherein, in Chemical Formula 1,
Ar₁ to Ar₄ are each independently a substituted or unsubstituted aryl group or a substituted or unsubstituted heteroaryl group,
at least one of Ar₁ to Ar₄ is a substituted or unsubstituted aryl group having 10 or more ring carbon atoms or a substituted or unsubstituted heteroaryl group having 10 or more ring carbon atoms.
R₁ to R₁₀ are each independently a hydrogen atom, a substituted or unsubstituted aryl group, a substituted or unsubstituted heteroaryl group, a substituted or unsubstituted alkyl group, a substituted or unsubstituted alkoxy group, a halogen atom, an electron withdrawing group, or a deuterium atom, and
adjacent ones of R₁ to R₁₀ are separate or are combined to each other to form a saturated or unsaturated ring.

8. The organic electroluminescence device as claimed in claim 7, wherein the aryl group having 10 or more ring carbon atoms includes a biphenyl group, a naphthyl group, a phenanthrenyl group, or a triphenylenyl group.

9. The organic electroluminescence device as claimed in claim 8, wherein the aryl group having 10 or more ring carbon atoms includes a biphenyl group.

10. The organic electroluminescence device as claimed in claim 7, wherein the heteroaryl group having 10 or more ring carbon atoms includes a dibenzohetero group.

11. The organic electroluminescence device as claimed in claim 10, wherein the dibenzohetero group is a group represented by the following Chemical Formula 2: 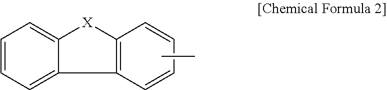
wherein, in Chemical Formula 2,
X is O, S, or Si(R₁₁)₂, and
R₁₁ is a substituted or unsubstituted alkyl group or a substituted or unsubstituted aryl group.

12. The organic electroluminescence device as claimed in claim 7, wherein Ar₁ to Ar₄ are each independently a substituted or unsubstituted aryl group having 10 or more ring carbon atoms or a substituted or unsubstituted heteroaryl group having 10 or more ring carbon atoms.

13. The organic electroluminescence device as claimed in claim 7, wherein the compound for an organic electroluminescence device includes one of the following Compounds 1-1 to 1-24:

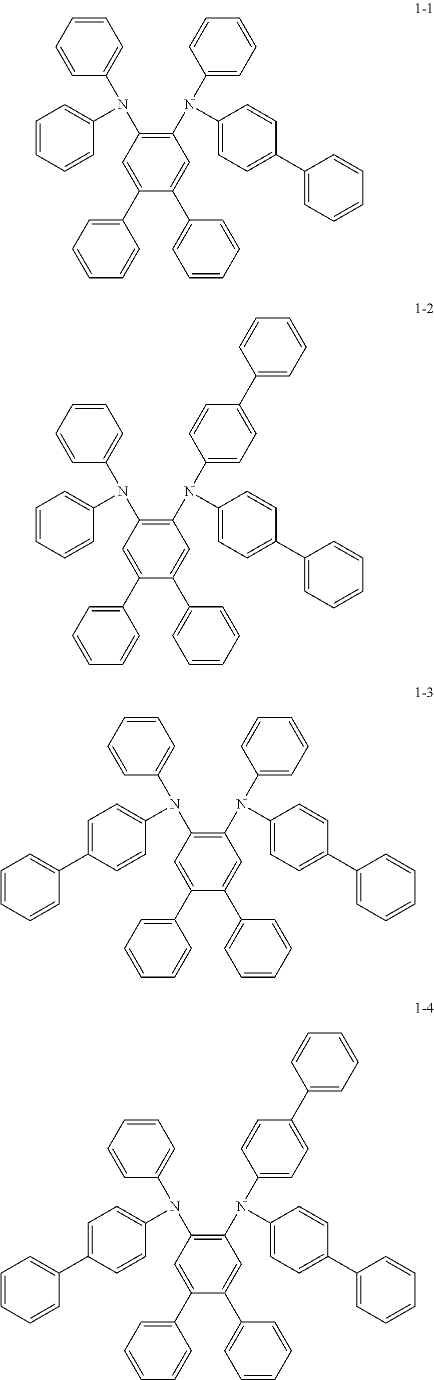 [SMILES: c1ccc(-c2ccc(N(c3ccccc3)c3cc(-c4ccccc4)c(-c4ccccc4)cc3N(c3ccccc3)c3ccccc3)cc2)cc1 ; c1ccc(-c2ccc(N(c3ccc(-c4ccccc4)cc3)c3cc(-c4ccccc4)c(-c4ccccc4)cc3N(c3ccccc3)c3ccccc3)cc2)cc1 ; c1ccc(-c2ccc(N(c3ccccc3)c3cc(-c4ccccc4)c(-c4ccccc4)cc3N(c3ccccc3)c3ccc(-c4ccccc4)cc3)cc2)cc1 ; c1ccc(-c2ccc(N(c3ccccc3)c3cc(-c4ccccc4)c(-c4ccccc4)cc3N(c3ccc(-c4ccccc4)cc3)c3ccc(-c4ccccc4)cc3)cc2)cc1]

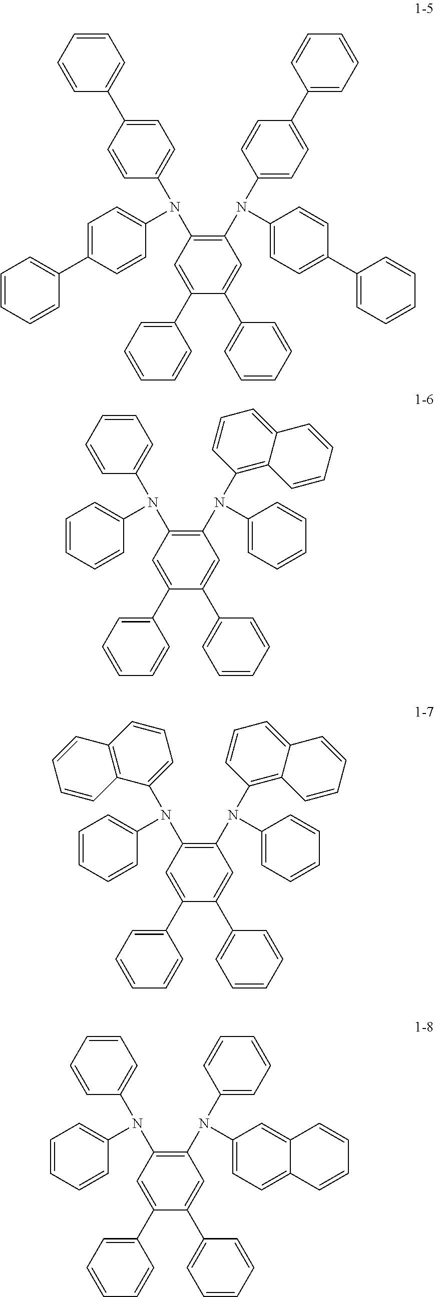

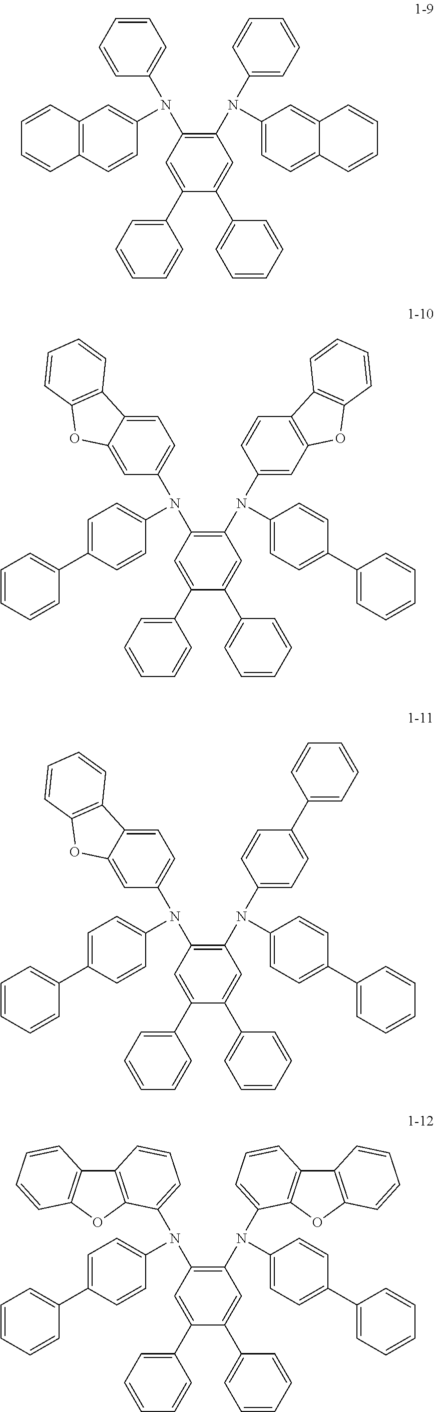 [SMILES: c1ccc(-c2cc(N(c3ccccc3)c3ccc4ccccc4c3)c(N(c3ccccc3)c3ccc4ccccc4c3)cc2-c2ccccc2)cc1 ; c1ccc(-c2ccc(N(c3ccc4c(c3)oc3ccccc34)c3cc(-c4ccccc4)c(-c4ccccc4)cc3N(c3ccc(-c4ccccc4)cc3)c3ccc4c(c3)oc3ccccc34)cc2)cc1 ; c1ccc(-c2ccc(N(c3ccc(-c4ccccc4)cc3)c3cc(-c4ccccc4)c(-c4ccccc4)cc3N(c3ccc(-c4ccccc4)cc3)c3ccc4c(c3)oc3ccccc34)cc2)cc1 ; c1ccc(-c2ccc(N(c3cc(-c4ccccc4)c(-c4ccccc4)cc3N(c3ccc(-c4ccccc4)cc3)c3cccc4c3oc3ccccc34)c3cccc4c3oc3ccccc34)cc2)cc1]

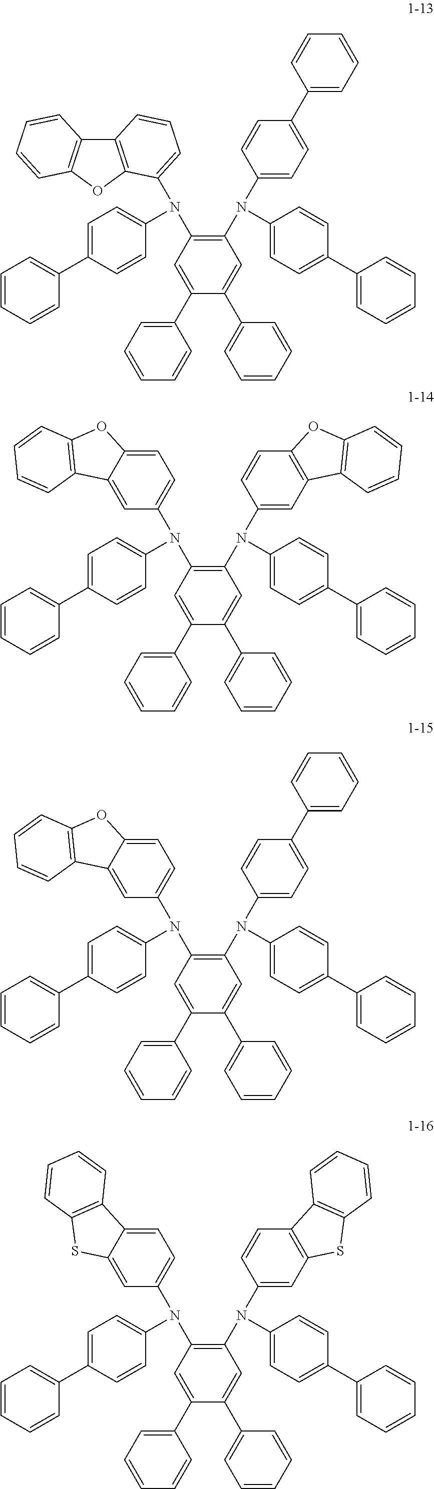 [SMILES: c1ccc(-c2ccc(N(c3ccc(-c4ccccc4)cc3)c3cc(-c4ccccc4)c(-c4ccccc4)cc3N(c3ccc(-c4ccccc4)cc3)c3cccc4c3oc3ccccc34)cc2)cc1 ; c1ccc(-c2ccc(N(c3ccc4oc5ccccc5c4c3)c3cc(-c4ccccc4)c(-c4ccccc4)cc3N(c3ccc(-c4ccccc4)cc3)c3ccc4oc5ccccc5c4c3)cc2)cc1 ; c1ccc(-c2ccc(N(c3ccc(-c4ccccc4)cc3)c3cc(-c4ccccc4)c(-c4ccccc4)cc3N(c3ccc(-c4ccccc4)cc3)c3ccc4oc5ccccc5c4c3)cc2)cc1 ; c1ccc(-c2ccc(N(c3ccc4c(c3)sc3ccccc34)c3cc(-c4ccccc4)c(-c4ccccc4)cc3N(c3ccc(-c4ccccc4)cc3)c3ccc4c(c3)sc3ccccc34)cc2)cc1]

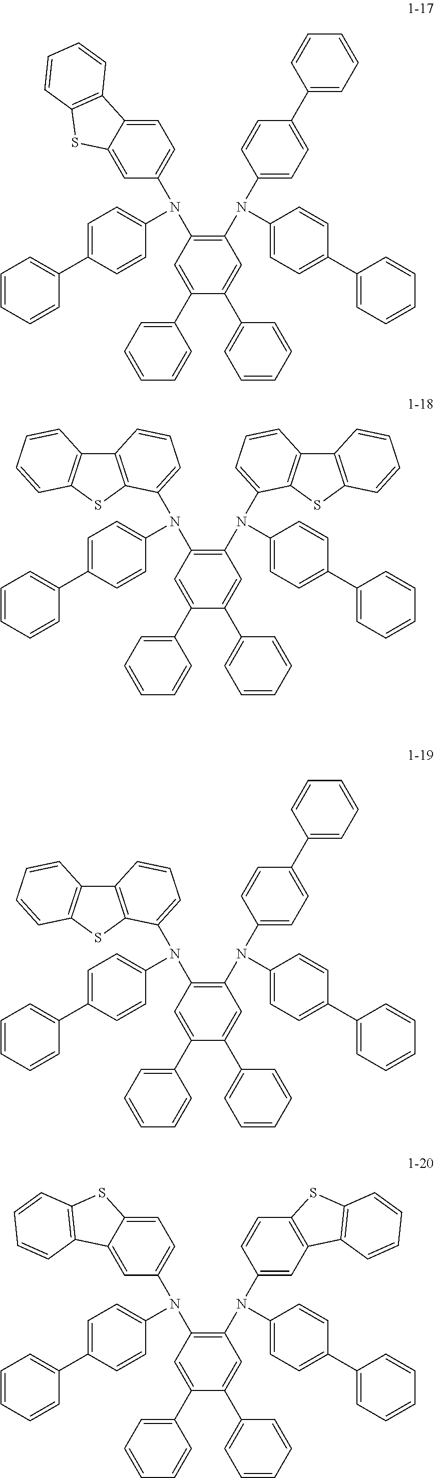 [SMILES: c1ccc(-c2ccc(N(c3ccc(-c4ccccc4)cc3)c3cc(-c4ccccc4)c(-c4ccccc4)cc3N(c3ccc(-c4ccccc4)cc3)c3ccc4c(c3)sc3ccccc34)cc2)cc1 ; c1ccc(-c2ccc(N(c3cc(-c4ccccc4)c(-c4ccccc4)cc3N(c3ccc(-c4ccccc4)cc3)c3cccc4c3sc3ccccc34)c3cccc4c3sc3ccccc34)cc2)cc1 ; c1ccc(-c2ccc(N(c3ccc(-c4ccccc4)cc3)c3cc(-c4ccccc4)c(-c4ccccc4)cc3N(c3ccc(-c4ccccc4)cc3)c3cccc4c3sc3ccccc34)cc2)cc1 ; c1ccc(-c2ccc(N(c3ccc4sc5ccccc5c4c3)c3cc(-c4ccccc4)c(-c4ccccc4)cc3N(c3ccc(-c4ccccc4)cc3)c3ccc4sc5ccccc5c4c3)cc2)cc1]

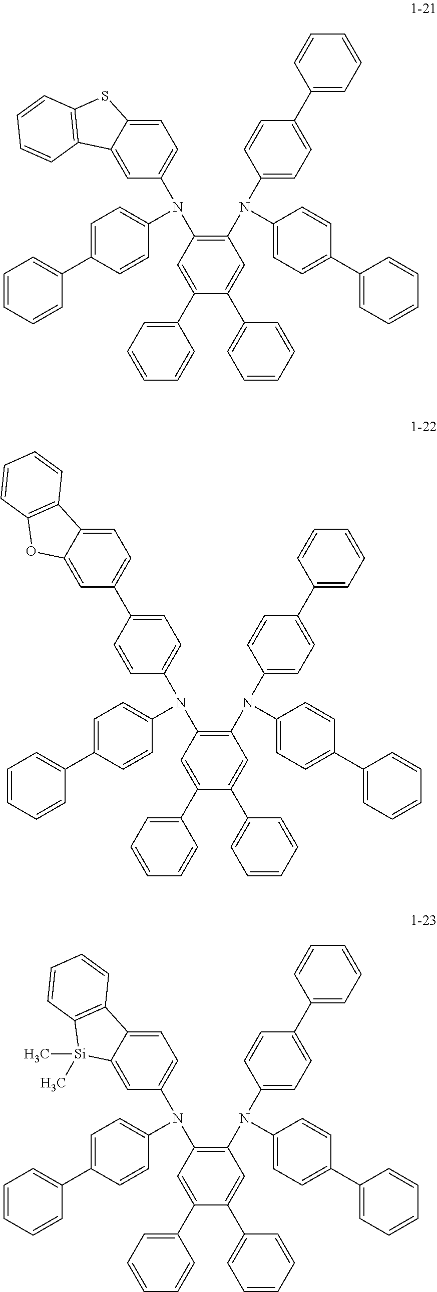 [SMILES: c1ccc(-c2ccc(N(c3ccc(-c4ccccc4)cc3)c3cc(-c4ccccc4)c(-c4ccccc4)cc3N(c3ccc(-c4ccccc4)cc3)c3ccc4sc5ccccc5c4c3)cc2)cc1 ; c1ccc(-c2ccc(N(c3ccc(-c4ccccc4)cc3)c3cc(-c4ccccc4)c(-c4ccccc4)cc3N(c3ccc(-c4ccccc4)cc3)c3ccc(-c4ccc5c(c4)oc4ccccc45)cc3)cc2)cc1 ; C[Si]1(C)c2ccccc2-c2ccc(N(c3ccc(-c4ccccc4)cc3)c3cc(-c4ccccc4)c(-c4ccccc4)cc3N(c3ccc(-c4ccccc4)cc3)c3ccc(-c4ccccc4)cc3)cc21]

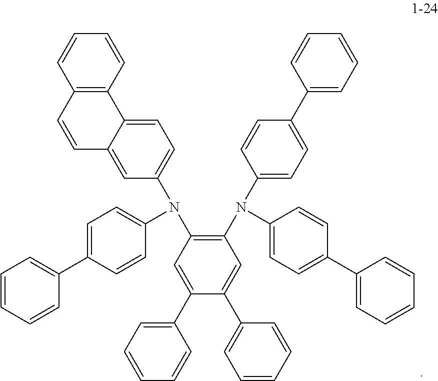 [SMILES: c1ccc(-c2ccc(N(c3ccc(-c4ccccc4)cc3)c3cc(-c4ccccc4)c(-c4ccccc4)cc3N(c3ccc(-c4ccccc4)cc3)c3ccc4c(ccc5ccccc54)c3)cc2)cc1]

14. The organic electroluminescence device as claimed in claim 7, wherein the compound for an organic electroluminescence device includes one of the following Compounds 1-25 to 1-44:

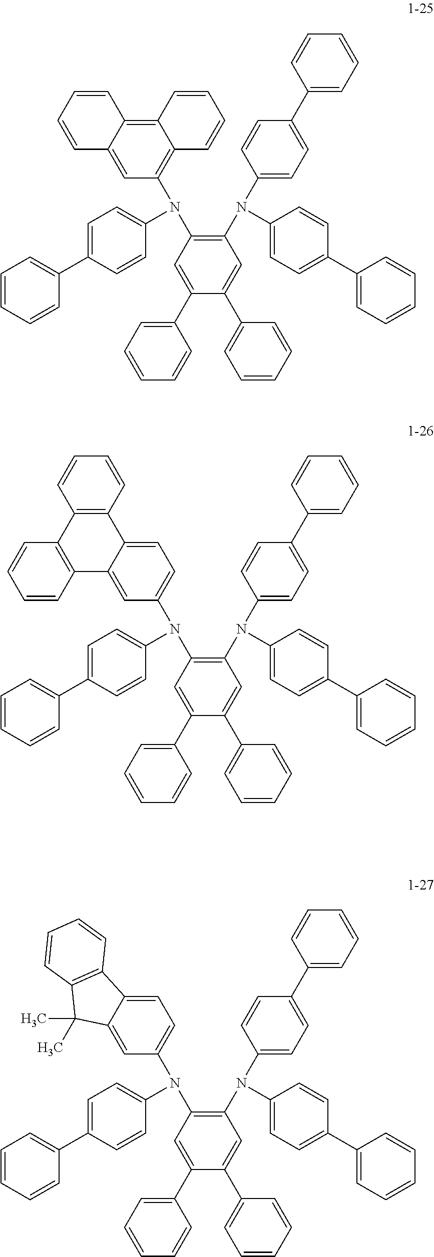 [SMILES: c1ccc(-c2ccc(N(c3ccc(-c4ccccc4)cc3)c3cc(-c4ccccc4)c(-c4ccccc4)cc3N(c3ccc(-c4ccccc4)cc3)c3cc4ccccc4c4ccccc34)cc2)cc1 ; c1ccc(-c2ccc(N(c3ccc(-c4ccccc4)cc3)c3cc(-c4ccccc4)c(-c4ccccc4)cc3N(c3ccc(-c4ccccc4)cc3)c3ccc4c5ccccc5c5ccccc5c4c3)cc2)cc1 ; CC1(C)c2ccccc2-c2ccc(N(c3ccc(-c4ccccc4)cc3)c3cc(-c4ccccc4)c(-c4ccccc4)cc3N(c3ccc(-c4ccccc4)cc3)c3ccc(-c4ccccc4)cc3)cc21]

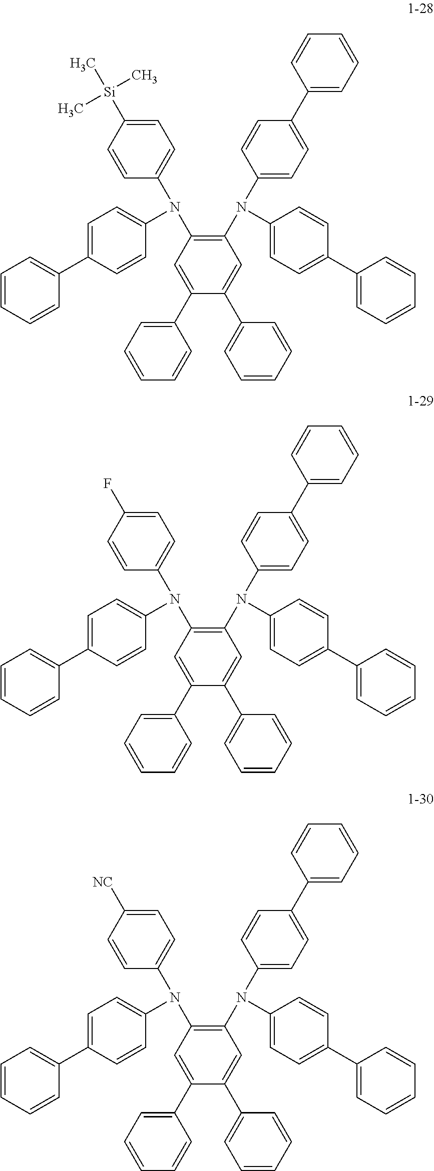 [SMILES: C[Si](C)(C)c1ccc(N(c2ccc(-c3ccccc3)cc2)c2cc(-c3ccccc3)c(-c3ccccc3)cc2N(c2ccc(-c3ccccc3)cc2)c2ccc(-c3ccccc3)cc2)cc1 ; Fc1ccc(N(c2ccc(-c3ccccc3)cc2)c2cc(-c3ccccc3)c(-c3ccccc3)cc2N(c2ccc(-c3ccccc3)cc2)c2ccc(-c3ccccc3)cc2)cc1 ; N#Cc1ccc(N(c2ccc(-c3ccccc3)cc2)c2cc(-c3ccccc3)c(-c3ccccc3)cc2N(c2ccc(-c3ccccc3)cc2)c2ccc(-c3ccccc3)cc2)cc1]

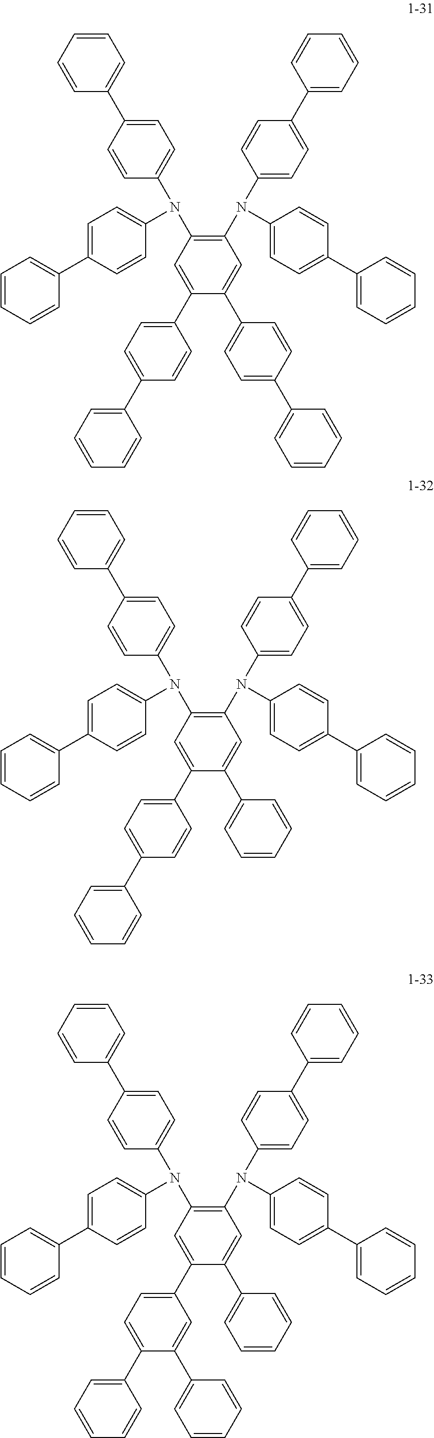 [SMILES: c1ccc(-c2ccc(-c3cc(N(c4ccc(-c5ccccc5)cc4)c4ccc(-c5ccccc5)cc4)c(N(c4ccc(-c5ccccc5)cc4)c4ccc(-c5ccccc5)cc4)cc3-c3ccc(-c4ccccc4)cc3)cc2)cc1 ; c1ccc(-c2ccc(-c3cc(N(c4ccc(-c5ccccc5)cc4)c4ccc(-c5ccccc5)cc4)c(N(c4ccc(-c5ccccc5)cc4)c4ccc(-c5ccccc5)cc4)cc3-c3ccccc3)cc2)cc1 ; c1ccc(-c2ccc(N(c3ccc(-c4ccccc4)cc3)c3cc(-c4ccccc4)c(-c4ccc(-c5ccccc5)c(-c5ccccc5)c4)cc3N(c3ccc(-c4ccccc4)cc3)c3ccc(-c4ccccc4)cc3)cc2)cc1]

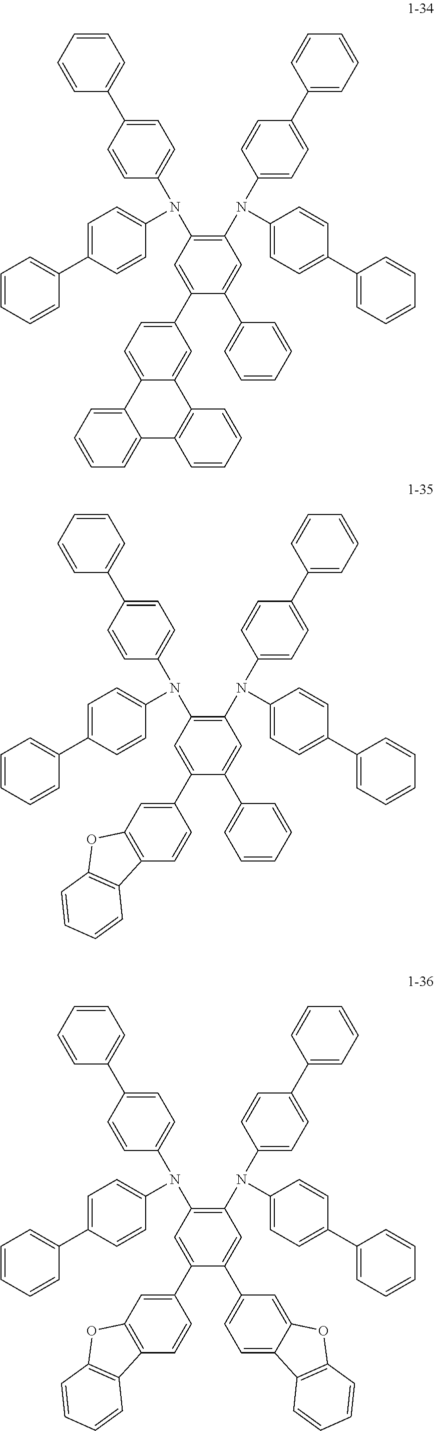 [SMILES: c1ccc(-c2ccc(N(c3ccc(-c4ccccc4)cc3)c3cc(-c4ccccc4)c(-c4ccc5c6ccccc6c6ccccc6c5c4)cc3N(c3ccc(-c4ccccc4)cc3)c3ccc(-c4ccccc4)cc3)cc2)cc1 ; c1ccc(-c2ccc(N(c3ccc(-c4ccccc4)cc3)c3cc(-c4ccccc4)c(-c4ccc5c(c4)oc4ccccc45)cc3N(c3ccc(-c4ccccc4)cc3)c3ccc(-c4ccccc4)cc3)cc2)cc1 ; c1ccc(-c2ccc(N(c3ccc(-c4ccccc4)cc3)c3cc(-c4ccc5c(c4)oc4ccccc45)c(-c4ccc5c(c4)oc4ccccc45)cc3N(c3ccc(-c4ccccc4)cc3)c3ccc(-c4ccccc4)cc3)cc2)cc1]

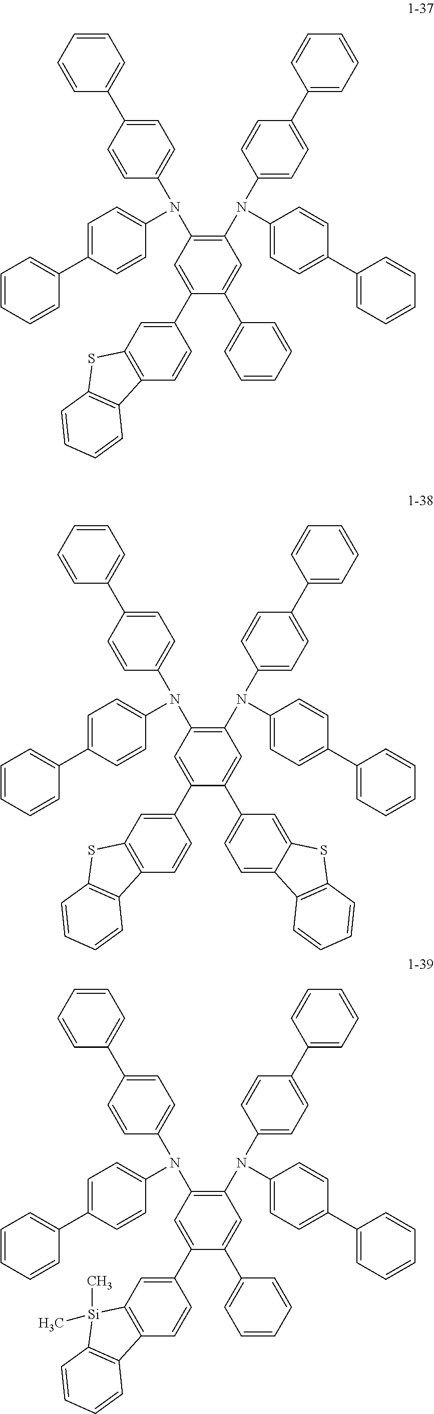

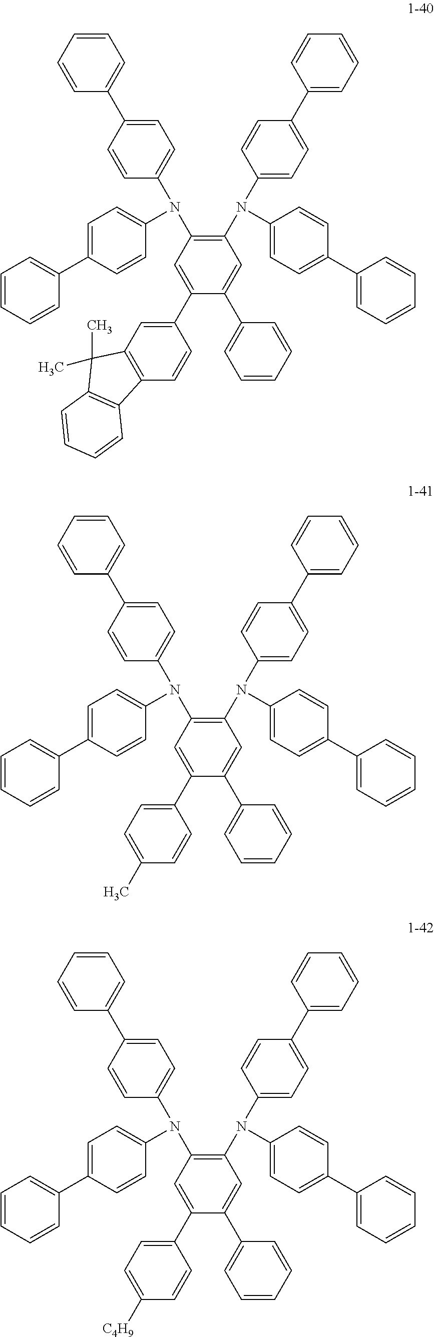

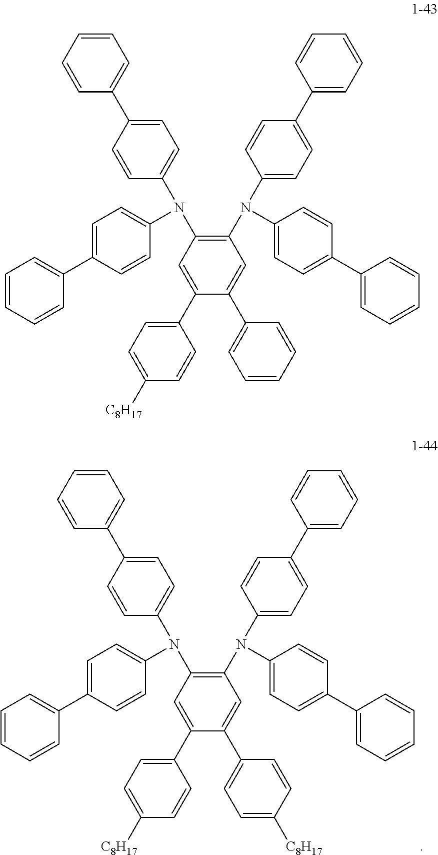

15. The organic electroluminescence device as claimed in claim 7, wherein the compound for an organic electroluminescence device includes one of the following Compounds 1-45 to 1-58:

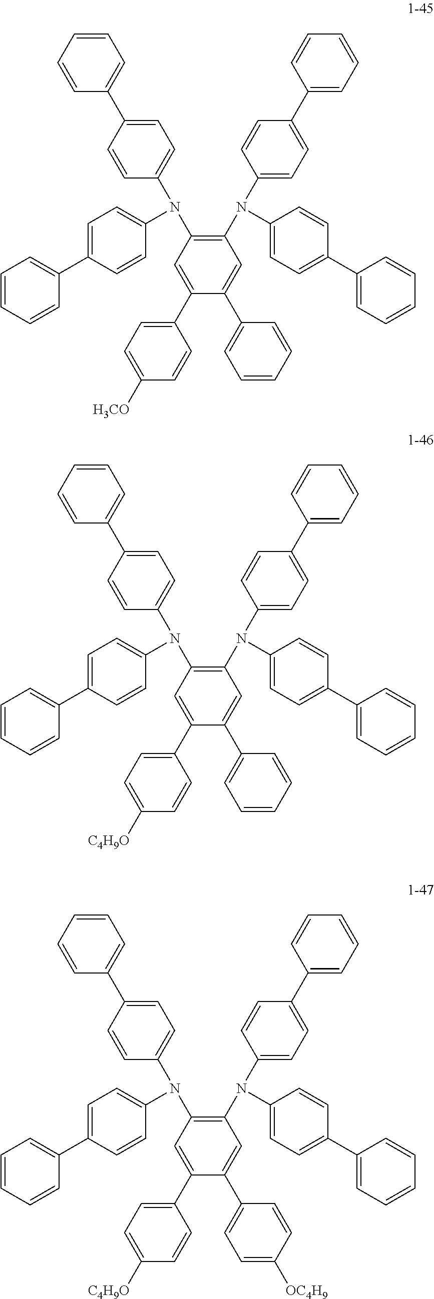

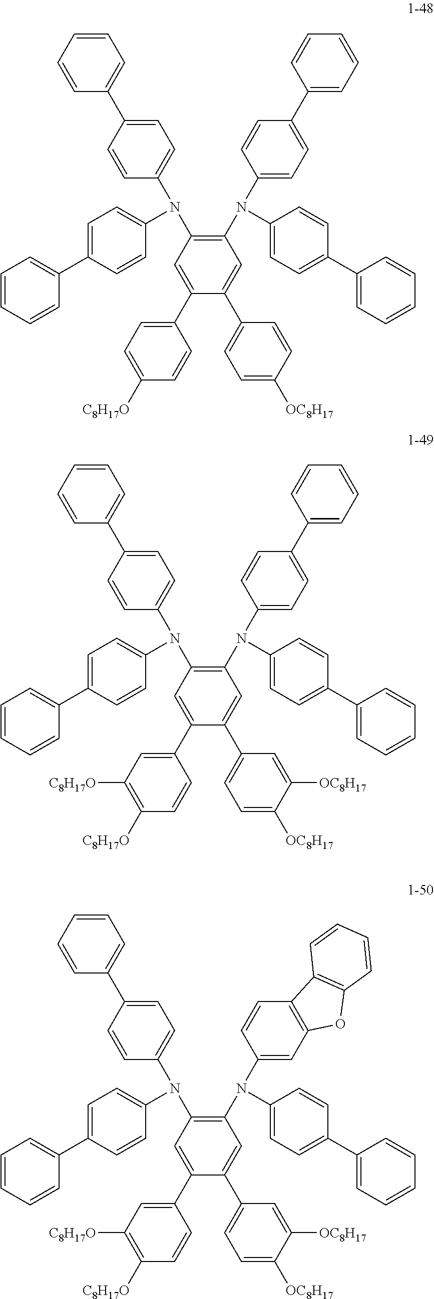

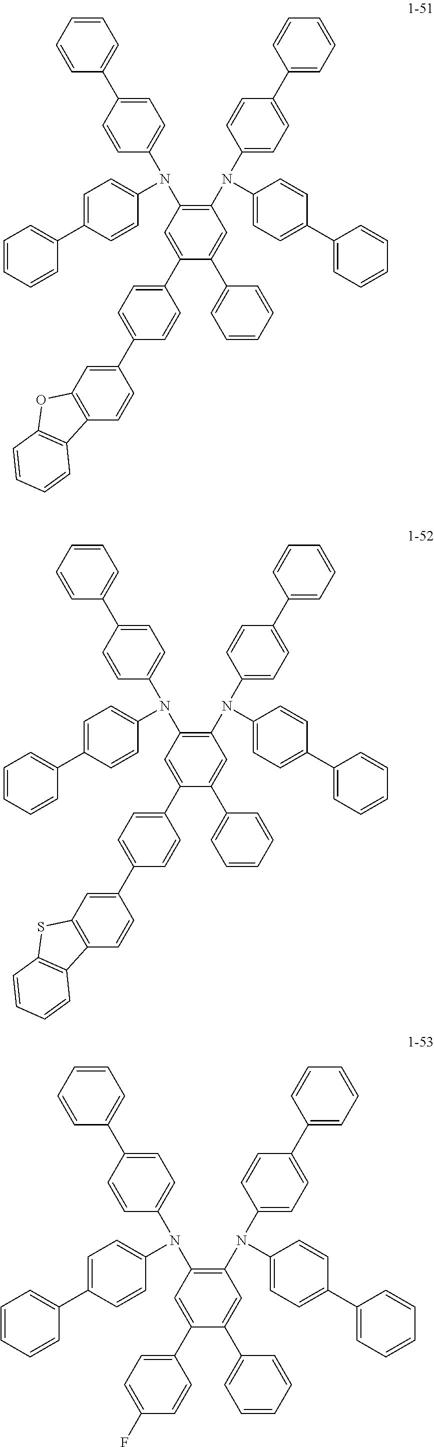 [SMILES: c1ccc(-c2ccc(N(c3ccc(-c4ccccc4)cc3)c3cc(-c4ccccc4)c(-c4ccc(-c5ccc6c(c5)oc5ccccc56)cc4)cc3N(c3ccc(-c4ccccc4)cc3)c3ccc(-c4ccccc4)cc3)cc2)cc1 ; c1ccc(-c2ccc(N(c3ccc(-c4ccccc4)cc3)c3cc(-c4ccccc4)c(-c4ccc(-c5ccc6c(c5)sc5ccccc56)cc4)cc3N(c3ccc(-c4ccccc4)cc3)c3ccc(-c4ccccc4)cc3)cc2)cc1 ; Fc1ccc(-c2cc(N(c3ccc(-c4ccccc4)cc3)c3ccc(-c4ccccc4)cc3)c(N(c3ccc(-c4ccccc4)cc3)c3ccc(-c4ccccc4)cc3)cc2-c2ccccc2)cc1]

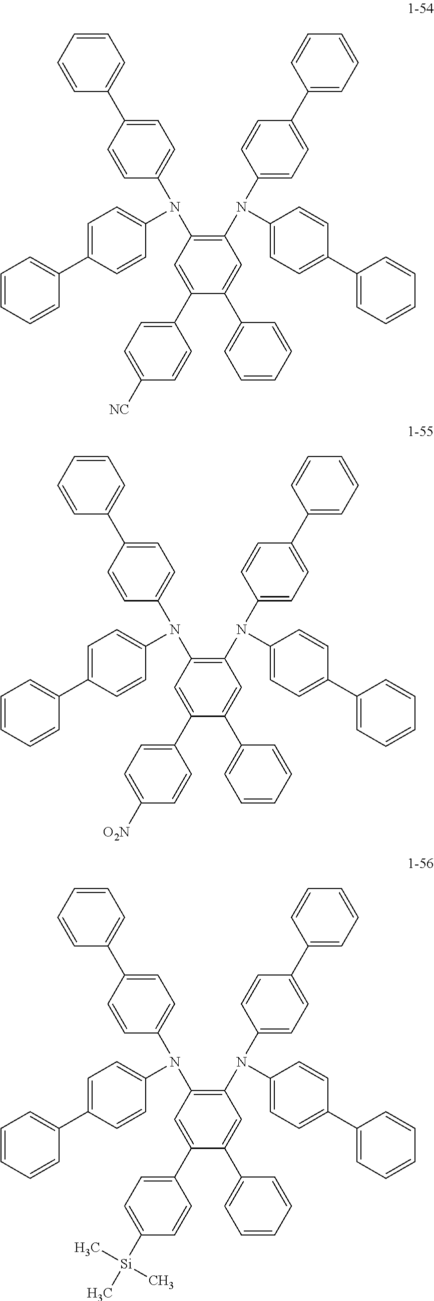 [SMILES: N#Cc1ccc(-c2cc(N(c3ccc(-c4ccccc4)cc3)c3ccc(-c4ccccc4)cc3)c(N(c3ccc(-c4ccccc4)cc3)c3ccc(-c4ccccc4)cc3)cc2-c2ccccc2)cc1 ; O=[N+]([O-])c1ccc(-c2cc(N(c3ccc(-c4ccccc4)cc3)c3ccc(-c4ccccc4)cc3)c(N(c3ccc(-c4ccccc4)cc3)c3ccc(-c4ccccc4)cc3)cc2-c2ccccc2)cc1 ; C[Si](C)(C)c1ccc(-c2cc(N(c3ccc(-c4ccccc4)cc3)c3ccc(-c4ccccc4)cc3)c(N(c3ccc(-c4ccccc4)cc3)c3ccc(-c4ccccc4)cc3)cc2-c2ccccc2)cc1]

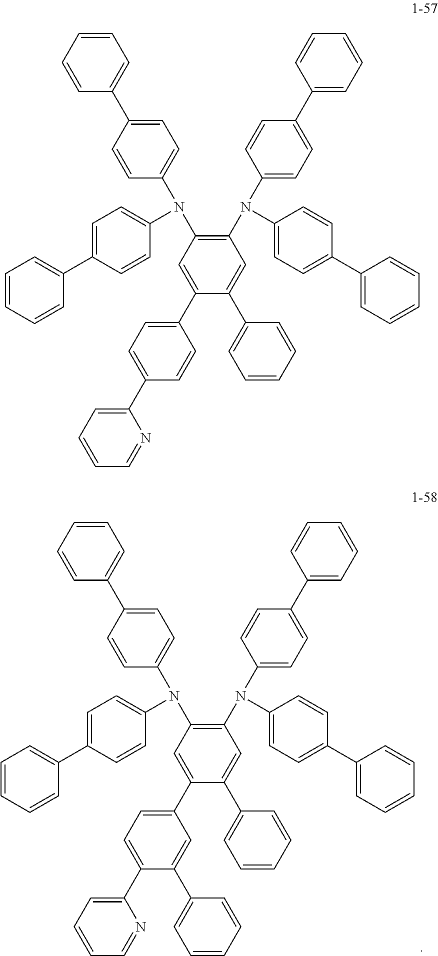 [SMILES: c1ccc(-c2ccc(N(c3ccc(-c4ccccc4)cc3)c3cc(-c4ccccc4)c(-c4ccc(-c5ccccn5)cc4)cc3N(c3ccc(-c4ccccc4)cc3)c3ccc(-c4ccccc4)cc3)cc2)cc1 ; c1ccc(-c2ccc(N(c3ccc(-c4ccccc4)cc3)c3cc(-c4ccccc4)c(-c4ccc(-c5ccccn5)c(-c5ccccc5)c4)cc3N(c3ccc(-c4ccccc4)cc3)c3ccc(-c4ccccc4)cc3)cc2)cc1]

16. The organic electroluminescence device as claimed in claim 7, wherein:
the organic electroluminescence device includes a hole transport layer, and
the compound for an organic electroluminescence device is included in the hole transport layer.
